(19) United States
(12) Patent Application Publication
(10) Pub. No.: US 2015/0141277 A1
(43) Pub. Date: May 21, 2015

(54) METHODS OF QUANTIFYING OF NUCLEIC ACIDS CAPTURED ON A SOLID SUPPORT

(71) Applicant: OXFORD TECHNOLOGY LIMITED, Begbroke (GB)

(72) Inventors: Edwin Southern, Begbroke (GB); Dietrich Lueerssen, Begbroke (GB); Oliver Miller, Begbroke (GB); Natalie Milner, Begbroke (GB)

(21) Appl. No.: 14/401,823

(22) PCT Filed: May 17, 2013
(86) PCT No.: PCT/GB2013/051289

§ 371 Date: Nov. 17, 2014

(30) Foreign Application Priority Data
May 17, 2012 (GB) 1208726.8

(51) Int. Cl. C12Q 1/68; G01N 27/447; G06F 19/00; G06F 19/20

(52) U.S. Cl. CPC C12Q 1/6834; C12Q 1/6816; G06F 19/20; G01N 27/447; G06F 19/3431
USPC 506/9; 435/6.11; 536/24.31; 204/451; 702/19

(57) ABSTRACT

A method for the measurement of the amount or difference in the amounts of 2 or more nucleic acid targets in a sample, the method comprising the steps of attaching to nucleic acids present in the sample (1) a tag which allows the nucleic acids to be captured to a solid support; and (2) a labelled probe for a first nucleic acid target present in the sample and a labelled probe for second nucleic acid target present in the sample, and then measuring the amount of each labelled probe or difference in the amount of labelled probes; wherein the probe is not a single labelled nucleotide.

The present invention relates to methods for detection of nucleic acid targets and materials for use in that method.

### BACKGROUND

Certain disorders and diseases are characterised by the presence of nucleic acid species in different amounts to those found in normal individuals. The present invention relates to methods and apparatus for the analysis of nucleic acid in individuals that may be indicative of the presence of a disorder or disease.

### STATEMENTS OF INVENTION

The invention relates to:

A method for the measurement of the differences in the amounts of 2 or more nucleic acid targets in a sample, the method comprising the steps of attaching to nucleic acids present in the sample
(1) a tag which allows the nucleic acids to be captured to a solid support; and
(2) a labelled probe for a first nucleic acid target present in the sample and a labelled probe for a second nucleic acid target present in the sample, and then
measuring the amount of each labelled probe or difference in the amount of labelled probes;
wherein the probe is not a single labelled nucleotide.

A method for the diagnosis of a nucleic acid imbalance associated with a disorder, the method comprising the steps of attaching to nucleic acids present in the sample
(1) a tag which allows the nucleic acids to be captured to a solid support; and
(2) a labelled probe for a first nucleic acid target present in the sample and a labelled probe for a second nucleic acid target present in the sample, and then
measuring the amount of each labelled probe or difference in the amount of labelled probes,
wherein detection of a relative difference between the amount of first and second target is indicative of the disorder, and wherein the probe is not a single labelled nucleotide.

A kit comprising a probe or probe set for a first nucleic acid and probe or probe set for a second nucleic acid, wherein the first probe or probe set is for a nucleic acid target associated with aneuploidy and a second probe or probe set is for a nucleic acid target not associated with aneuploidy.

A kit comprising a probe or probe set for a first nucleic acid and probe or probe set for a second nucleic acid, wherein the first probe or probe set is for a nucleic acid target associated with a disorder and a second probe or probe set is for a nucleic acid target not associated with the disorder, wherein the disorder is associated with a change in the amount of the first nucleic acid target in a genome.

A kit comprising a tag that may be attached to a nucleic acid to allow that nucleic acids to be captured to a solid support and a probe or probe set for a nucleic acid target associated with a disorder, the disorder being associated with a change in the amount of nucleic acid target in a genome, such as aneuploidy.

### FIGURES

FIG. 1: Plot of total integrated signal intensity for each sample on the capture slide

FIG. 2: Plot of mean total integrated signal intensity and standard deviation of the replicates

FIG. 3: The layout of the samples on the capture lawn for example 8.2

FIG. 4: The scanned image of data from example 8.2

FIG. 5: A plot of the total integrated signal intensity of each sample for example 8.2

FIG. 6: The layout of the samples on the capture lawn for example 8.3 (10% fetal DNA)

FIG. 7: The scanned image for example 8.3 (10% fetal DNA)

FIG. 8: Local background calculation

FIG. 9: Ratios of the total integrated signal intensities of the 635 and 532 labelled RNA probe:tagged genomic hybrids at 10% modelled fetal content. Trisomies in both the 532 and 635 libraries are modelled and compared to the disomy ratio. Both the raw and local background subtracted data are shown

FIG. 10: Z scores of each of the 10% modelled fetal content samples calculated from the mean and standard deviation of the disomy samples. Also shown are Z scores generated from MPSS.
Figures show massively parallel sequencing normalised chromosome values compared with karyotype classifications for chromosomes 21, 18, and 13. Circles display classifications for chromosome 21, squares display classifications for chromosome 18, and triangles display classifications for chromosome 13. Unclassified samples with trisomy karyotypes have been circled. Bianchi. Genome-Wide Fetal Aneuploidy Detection. Obstet Gynecol 2012.

FIG. 11: The layout of the samples on the capture lawn for example 8.3 (5% fetal DNA)

FIG. 12: The scanned image for example 8.3 (5% fetal DNA)

FIG. 13: Calculation of the local background values example 8.3 (5% fetal DNA)

FIG. 14: Ratios of the total integrated signal intensities of the 635 and 532 labelled RNA probe:tagged genomic hybrids at 5% modelled fetal content. Trisomies in both the 532 and 635 libraries are modelled and compared to the disomy ratio. The local background subtracted data is shown

FIG. 15: Z scores of each of the 5% modelled fetal content samples calculated from the mean and standard deviation of the disomy samples.

FIG. 16: Experimental design based on dye-swap

FIG. 17: Analysis profile for dye swap approach

FIG. 18: The layout of the samples on the capture lawn for example 10.

FIG. 19 Slides 1-27 supporting examples 1-5

FIG. 20 Slides 1-13 disclosing principles of microfluidic methodology

FIG. 21: Slides supporting Example 6

FIG. 22 Screen Tape analysis of tailed samples (Example 12)

FIG. 23 Slide layout (Example 12)

FIG. 24 Slide Image (Example 12)

FIG. 25 Mean of median raw integrated pixel intensities

FIG. 26 Calculation of the ratio of R-ratios (see Example 12)

FIG. 27 Cross-referencing the data points (see Example 12)

FIG. 28 Probability density function (example 12)

### DETAILED DESCRIPTION

The present invention relates generally to apparatus and methods for the measurement of differences in the amounts of two or more specific nucleic acids in a sample. The method comprises attaching to nucleic acids present in the sample
(1) a tag which allows the nucleic acids to be captured to a solid support; and
(2) a labelled probe for a first nucleic acid target present in the sample and a labelled probe for a second nucleic acid target present in the sample, and then
measuring the amount of each labelled probe or difference in the amount of labelled probes;
wherein the probe is not a single labelled nucleotide.

The sample may be from any species, such as non-human animal, plant or prokarycyte or human from which it is desired to assess the different levels of two nucleic acid species.

The nucleic acid source may be human, animal, plant, bacterial or viral, by way of non-limiting example.

The sample may comprise nucleic acid derived from the blood or urine of an individual being assessed for a disease state or condition, or being assessed for the condition of a fetus.

In particular the sample may comprise nucleic acid derived from the blood or urine of a pregnant female, such as a female in the first trimester of pregnancy. The blood and urine of pregnant women comprises circulating fetal DNA, and this DNA may be used in non invasive prenatal diagnostics (NIPD). For example, where the fetus has an abnormally high number of copies of a chromosome, such as chromosome 21, detection of the additional levels of that chromosome in the blood of the mother can allow diagnosis of the aneuploidy of the fetus.

Therefore the invention also provides a method for the diagnosis of a nucleic acid imbalance associated with a disorder, the method comprising the steps of attaching to nucleic acids present in the sample
(1) a tag which allows the nucleic acids to be captured to a solid support; and
(2) a labelled probe for a first nucleic acid target present in the sample and a labelled probe for a second nucleic acid target present in the sample, and then
measuring the amount of each labelled probe or difference in the amount of labelled probes,
wherein detection of a relative difference between the amount of first and second target is indicative of the disorder, wherein the probe is not a single labelled nucleotide.

The sample may also comprise nucleic acid derived from the blood or urine or other source of an individual being assessed for presence or development of a disease associated with an increased or decreased amount of a target nucleic acid , for example cancer. For example, certain cancers are associated with an increased or decreased amount of a circulating nucleic acid diagnostic of that cancer.

The sample may also comprise nucleic acid derived from the blood or urine of an individual who has received a donor organ. The analysis of donor organ nucleic acid in the bloodstream can be used to assess the risk of organ failure (see for example, http://www.nature.com/news/2011/110328/full/news.2011.189.html).

Measurement of nucleic acid present in the sample may also be taken to cover measurement of nucleic acid that may be present in the sample, and thus the invention covers the scenario where the presence of a target is not confirmed in the sample, and also where the target is known to be present.

In one aspect the nucleic acid in the sample is not size selected before use in the present invention.

Where the nucleic acid sample is from a pregnant female then in one aspect the nucleic acid in the sample is not treated so as to increase the relative percentage of fetal nucleic acid versus materially derived nucleic acid in the sample. Therefore in one aspect the sample exposed to the probe includes the, or substantially the, same ratio of fetal to maternal nucleic acid as is found in vivo in the pregnant female, acknowledging that some DNA extraction procedures may have a minor inherent bias to certain types of sequences. Generally the process of the invention is not designed to enrich for fetal nucleic acid.

The tag may be a nucleic acid species, for example, may be a homopolymer of nucleotides added by terminal transferase to an existing nucleic acid species in the sample, or may be an oligonucleotide differing in sequence from any sequence that is known or expected to be present in the nucleic acids of the sample. For example the tag may be a polyA tail, capable of being attached to a solid support having a poly T complement, or may be biotin or a similar moiety such as DSB-X (a low-affinity derivative of biotin) capable of attaching to streptavadin or avadin on a solid support. Suitably the tag is not specific for any nucleic acid present in the sample but can generally be attached to all the nucleic acids present in the sample.

In one aspect the tag is covalently attached to the nucleic acids of the sample.

In one aspect the tag is not selective for nucleic acid sequences in the target.

In one aspect the tag may be (e.g. a homopolmer) of a defined length or be within a defined range of fragment lengths. For example, nucleic acid may be labeled with e.g. a poly A tail or other suitable polynucleotide tail in a reaction that is stopped after a defined time before the tail reaches the maximum tail length. In one aspect a polyA tail added to a nucleic acid from a sample may be less than 1000 nt, such as between 50-800 nucleotides, such as between 50-600 nucleotides, such as 50-500 nucleotides, such as 100-500 nucleotides or 200-400 nucleotides in length, which may be achieved for example by the addition of defined polyA tails or termination of a polyA tailing reaction at an appropriate time to produce tails of desired length.

The present invention is distinguished from sandwich hybridisation, in which the presence of foreign nucleic acids is measured by hybridisation to a solid support and detection via hybridisation of a labelled probe. In sandwich hybridisation capture is selective and sequence specific; and capture is mediated by a separate oligonucleotide molecule that is not covalently attached to the target molecule. The oligonucleotide molecule comprises two regions; a sequence-specific target capture region and a homopolymer solid support capture region. It is intended that all captured molecules are also hybridised with a labelled DNA oligonucleotide probe for detection. In contrast, in the method presented here, target molecules are suitably modified via covalent attachment of a tag that is intended for non-selective capture of all sequences.

In one aspect the first and second nucleic acid targets are located on different chromosomes. In one aspect the first probe may be for a target nucleic acid sequence associated with aneuploidy and the second probe is for target nucleic acid not generally associated with aneuploidy. For example, the first probe may be for human chromosome 21, 13, or 18, which are associated with Downs syndrome, Patau syndrome or Edwards syndrome, respectively. Aneuploidy may be, for example, trisomy or monosomy. The second probe may be to a chromosome which is not associated with aneuploidy in adult humans, such as chromosome 1. The probe for the second nucleic acid in the sample suitably provides an internal control, for example can provide a level for the amount of a nucleic acid in a diploid chromosome which can be compared with the amount of a nucleic acid in a possibly trisomy.

In one aspect the first probe is for a target nucleic acid sequence associated with a cancer and the second probe is for target nucleic acid not associated with cancer. In one aspect the first probe is for a target nucleic acid sequence, the increase or decrease of that target in the genome relative to a normal amount being associated with a disease or disorder, and the second probe is for target nucleic acid not associated with the disease or disorder.

In one aspect the first probe is for a target nucleic acid sequence associated with a specific mRNA and the second probe is for target nucleic acid not associated with that mRNA.

In one aspect the first target is a first chromosome and the second target is a different chromosomal target.

The probe may be a nucleic acid such as DNA or RNA, or a modification thereof such as, but not limited to, a nucleic acid modified to change Tm e.g. increase or decrease Tm, or modified to change nuclease sensitivity e.g. in the form of e.g. locked nucleic acid and peptide nucleic acid, phosphorothioates or oligonucleotides comprising a O-Me linkage. Alternatively the probe could be a protein or polypeptide specific to nucleic acids, such as an antibody or fragment thereof, for example an antibody to a DNA or RNA sequence.

In one aspect of the invention a single probe to each target may be used. In another aspect of the invention a set of different probes for the same target may be used, for example to increase the sensitivity of the method. For example, where the method is used to detect aneuploidy, and the amounts of DNA of different chromosomes are being compared, then the use of a set of probes to a first chromosome target will provide information on that first target. Likewise a second set of probes can be used for a second target.

Therefore the invention relates to a method comprising use of a first set of labelled probes for a first nucleic acid target and a second set of labelled probes for a second nucleic acid target, wherein the first and second sets contain multiple different probes for the first and second nucleic acid targets respectively. In this aspect a target may be capable of binding to multiple probes.

Any reference to use of a probe herein may be taken to refer to a single probe for a target or, in a further aspect, may be taken also to refer to the use of a set of different probes (a probe set) for the same target, unless otherwise apparent from the context.

A probe set suitably comprises multiple specific probes for the target, such as a mixture of at least 2, 3, 4, 5, 10, 20, 30, 40, 50, 100, 250, 500, 750 or 1000 probes for a target (such as a chromosome), or even more.

Where a set of probes is used for a given target then each member of the set of probes suitably has the same label, such that measurement of one label across the whole sample reflects the total amount of target, even where there are multiple probes for that target.

The probes may be labelled with any suitable label, such as a radioactive or fluorescent label. In one aspect the first and second probes comprise fluorescent labels with distinguishable emission spectra and the amount of a probe, or the difference between the probes, is measured by fluorescence. Alternatively the amount of probe may be measured by determining the fluorescence lifetime. In a further aspect the Raman spectra of a probe or probe set may be measured, for example to determine the amount of probe.

As well as a first section of a probe that is specific for a target region, each probe may comprise a second section that may be used to identify or select the probe. Where a set of probes to a target is used, then each probe in the probe set may comprise a second section that is the same, or functions in the same way, across all members of the set. This region can suitably allow the members of a probe set to be identified and/or selected by, for example, hybridisation of a complement to the second region. The first and second probes, or each member of a set of probes, therefore may comprise a first section of sequence complementary to a target nucleic acid, and further comprise a second section that can be used to differentiate the first and second probes or to differentiate a first and second set of probes. In one aspect the sequences of the second sections of the olignucleotide reagents are chosen such that they would not form stable duplexes with any nucleic acid in the sample.

It will be appreciated that the invention is not limited to the use of two probes for two targets. Additional probes and targets may be used. For example a third probe or probe set might be used to probe the amount of a third target. That target might serve as a second control. Indeed, comparison between two controls might also be used to provide an internal control on the degree of error in the methodology. For example, in determination of aneuploidy on chromosome 21, three probes or probe sets, one to chromosome 21 and 2 probes or probe sets to different chromosomes not associated with aneuploidy could be employed.

Thus in a further aspect the method of the invention comprises a third probe comprising a third label, detectably different from the first or second labels. The ratio of label for target of interest to a first reference target, ref1, can be compared with the ratio of a second reference target, ref2, to ref1.

The probe is not a single labelled nucleotide. Suitably the probe is an oligonucleotide having at least 5, such as at least 10 nucleotides, for example between 5-100 nucleotides, such as 10-50 nucleotides, such as 10-30 nucleotides.

In a further aspect of the invention, a probe set need not contain probes specific to a single chromosome. Instead, it may be advantageous to design probe sets comprising probes specific to sequences on two or more chromosomes. For example, the reference probe set may comprise probes specific to sequences on multiple chromosomes. In another example, the probe set for the condition or disease may comprise probes specific to sequences from more than one chromosome.

It can be seen that, by way of non limiting example, a target may therefore be a single binding site for a single probe, or multiple sites for multiple probes on the same chromosome, or multiple sites for multiple probes on a subset of chromosomes.

In this aspect a first probe set 1 may target to N chromosomes and a second probe set 2 may target M chromosomes, where N+M is equal to or less than the number of autosomes, and the chromosomes targeted by probe set 1 and probe set 2 are different and non overlapping.

In one aspect a set of probes is designed to cover the whole genome (excluding X and Y chromosomes) such that each chromosome is represented by the same number of probes, and suitably having substantially the same GC coverage. The probes may be split into two sets of libraries with half of the chromosomes in one library and the other half in the other library. Each library is labelled with a different label (e.g. colour). A difference in ratio between the two labels (colours) would indicate aneuploidy. With a two colour system aneuploidy in a single chromosome would give a difference in signal of 4%.

In another one aspect a sex chromosome or chromosomes may be a part of the genome that is probed.

In another aspect sex chromosomes may optionally be labelled with a third label (eg colour).

In one aspect the measurement of probe hybridisation occurs at the level of individual probes. In essence the number of individual probes binding to a target may be counted.

In another aspect the measurement occurs across the population of labelled probes for the first target and across the population of probes of the second target. For example, where the probe or probe sets used for a target are labelled by a fluorescent marker, then the total florescence of a sample, or a defined part therefore (such as a defined volume of the sample) may be measured. Detection across a sample may have the advantage that there is no need for individual counting of each single probe-target attachment event.

The target nucleic acid of the invention is suitably captured onto a solid support using the tag attached to the nucleic acids. The solid support may be a bead or sheet such as a slide or plate, for example, of glass or plastic. The bead may be a column packing material, or magnetic bead. The solid support may also be the outside of a rod or the inside of a tube—such as an optical fibre or a glass pipette respectively. The bead or sheet material or rod or tube may be derivatised with a complement of the tag attached to the nucleic acids in the sample, and the solid support may be contacted with the sample to allow the tag to bind to its complement and thus attach to the solid support.

The probe and target may be attached to one another before the combination is contacted with the solid support. Alternatively the tagged nucleic acid may be contacted with a solid support after which the probe is contacted with the tagged nucleic acid on the solid support, to allow formation of the attachment of the probe and target.

Attachment of the tag and nucleic acid may be achieved using enzymatic extension of the nucleic acids, for example using terminal transferase. Attachment of the target and probe may be by nucleic acid complementary strand hybridisation. Suitable reaction conditions for the attachment of tags and probes are well known in the art. For example, where the probes are nucleic acid probes, then the target nucleic acids and the sets of labelled probes may be mixed in solution and allowed to hybridise under conditions suitable for nucleic acid hybridisation, which are well known in the art.

Where the probe is a single stranded nucleic acid then suitably the target nucleic acid is denatured before hybridisation with the probe.

In one aspect the probe and target are bound to a solid support, after which the non captured label is washed away to remove background label.

The amount of probe bound to the target may be measured in different ways, illustrated by the following different aspects.

In one aspect the amount of labelled probe is directly detected on the solid support.

For example, where the solid support is a sheet material and the label is a fluorophore, the area of sheet material to which the mixture of probe and target was applied may be analysed in a fluorescence scanner to measure the fluorescence of the fluorophores used to label the labelling probes.

In another aspect the labelled probe is eluted or in the case of nuclease resistant labelled probes, digested, using nucleases from the solid support and the label in the eluate is measured. This method may, for example, be used when the solid support is a particulate material such as a bead or a column packing material.

For example, the solid support may comprise a particulate material such as a bead or column packing material, and the complex formed between target and probe may be passed through a column comprising the particulate material or mixed with the beads, such as magnetic beads. The particulate material may be then washed to remove non-hybridised probes. Bound probes may be then eluted from the solid support and the label, of the eluate e.g. the fluorescence spectrum of a fluorescent label, is measured to quantify the relative amounts of different probes as a measure of the relative amounts of the target nucleic acids in the sample.

Where the probe-target complex is eluted from a solid support, in one aspect the amount of probe (and therefore target) in an eluate may be assessed by measurement of the distance that the elute flows across a lawn of capture molecules which are complementary to the probe, or any part of the target not bound to probe, before the signal is depleted.

For example, in one aspect probes may comprise oligonucleotides with one section of sequence complementary to targets whose amounts are to be compared, and a second section that is common to all members of a set representing a region(s) of each target in the sample. The probe and target may be hybridised and attached to a solid support. The non-captured oligonucleotide reagents may be washed away. The complex formed between the target nucleic acids and the labelled probes may be eluted under conditions which remove them from the support. The relative amounts of the labelled probes in the eluate may be determined by flowing them over a lawn of oligonucleotide capture agents attached to a solid support. The oligonucleotide capture agents comprise a mixture of oligonucleotides with sequences complementary to the sequences of each of the second sections of the labelled probes. Conditions are chosen such that the labelled oligonucleotide reagents are initially in molar excess over their complements in the lawn, so that as the mixture flows over the lawn, the labelled oligonucleotide reagents saturate their complements on the surface, until they have been depleted to a level such that they are not in molar excess over their complements. The point at which depletion below excess occurs depends on the concentration of the target in the sample—those in smallest amount are depleted first. The relative amounts of component targets in the sample may be measured by measuring the distance migrated of each of the fluorescent labels along the flow path.

In another aspect the amount of probe in an eluate may be assessed by measurement of the amount of eluate that is captured by a capture molecules complementary to the probe, or any part of the target not bound to probe, on a column.

For example, the eluate may be applied to a column having capture molecules under conditions which allow attachment between the second sections of the probes and the capture molecules on the solid support. Conditions may be chosen such that the labelled probes are initially in molar excess over their complements in the column, so that as the mixture flows through the column, the labelled probes saturate their complements on the support, until they have been depleted to a level such that they are not in molar excess over their complements. The point at which depletion below excess occurs depends on the concentration of the target in the sample—those in smallest amount are depleted first. The relative amounts of component targets in the sample may be measured by eluting the column under conditions which dissociate the probes from the column. The relative amounts of the components in the target may be measured from the relative amounts of the corresponding label—eg fluorophore, measured as the outflow from the column passes a detector, e.g. a fluorescence detector.

In the above aspect a probe having a second (non-target specific) section is used in which this second section acts as a target for a capture molecule. In another aspect the second section of the probe may alternatively or additionally act as a modulator of mobility during electrophoresis, for example capillary electrophoresis. This allows probes to be discriminated and hence allow quantitation of their corresponding nucleic acid targets.

Differential mobility may be conferred by oligonucleotide sections of different length or other moieties which confer different charge or different bulk.

In such a scenario labelling of probes or sets of probes can use a single fluorochrome or species of fluorophore, or alternatively two sets of probes for two different targets may be labelled with different labels which allows the amount of each to be distinguished. Preferred labels comprise the fluorescent labels used routinely in capillary sequencing.

Thus an aspect of the invention relates to a method wherein the second sections of the first and second probes are different from one another and the second section acts as a modulator of mobility during electrophoresis such that the first and second probes may be discriminated by differential mobility during electrophoresis.

By way of example, a probe- target complex may be formed and captured onto a solid support, followed by washing away of any non-captured oligonucleotide reagents. The complex is eluted under conditions which remove them from the solid support.

The relative amounts of the labelled oligonucleotide reagents may be determined by electrophoretic separation, for example, by capillary electrophoresis.

In a further aspect the method of the invention uses a pair of probes, wherein each probe has a first target-specific section and a second (non-target specific) section, and wherein the second sections of the pair of probes are complementary to one another. One of the pair of complementary probes comprises a label, such as a fluorophore, and the other a label which is a quenching agent for the first label, serving to remove or negate or mask the signal of the first label. For example the first label may be a fluorophore and the second label is a quenching agent for a fluorophore, such that the quenching of the fluorophore would be complete when the 2 different probes (and hence 2 different targets) were present in the same amount and the probes were attached to one another to allow the quenching of the signal of the first label. An imbalance of target would lead to incomplete quenching of the label when the label was in excess over the quenching agent.

Therefore a further aspect of the invention relates to a method wherein the second section of the first and second probes are complementary in sequence, such that they can hybridise with one another, and wherein the first and second probe are labelled with a fluorophore and with a quenching agent, respectively, such that hybridisation of the complementary sequences of the first and second probes brings the quencher and fluorophore into juxtaposition such that quenching of the fluorophore can take place on juxtaposed probes.

The invention also relates to a pair of probes, a first probe specific for one target nucleic acid sequence and a second probe specific for a second target nucleic acid sequence, wherein the first and second probes share a complementary sequence. The first probe may be labelled, e.g. with a fluorophore, suitably at one end of the second section of the probe. The second probe may be labelled with a quencher to the label, eg. a quencher to the fluorophore, at the other end of the second section of the probe.

Suitably the sequences of the second sections of the olignucleotide reagents are chosen such that they would not form stable duplexes with any nucleic acid in the sample.

For example, in one aspect a probe specific for the second member of the paired targets contain a second section which is complementary in sequence to the first member of the pair, which is expected to be in excess of or equivalent in amount to the second member of the pair of targets. The members of the first paired set are labelled, e.g. with a fluorophore at one end of the second section of the probe. The members of the second paired set are labelled with a quencher to the fluorophore at the other end of the second section of the tag.

In one aspect the target nucleic acids and the pair of labelled probes are mixed in solution and allowed to hybridise under conditions which allow duplex formation between the target specific sections of the tagged oligonucleotides, but not between the common sections of the probes.

In one aspect the complexes of probe and target may be captured onto a solid support which may be then washed to remove non-captured oligonucleotide reagents. The complex may be then eluted from the solid support. The complementary sections of the probes in the eluate are then allowed to hybridise, bringing the quencher and fluorophore into juxtaposition.

A fluorescence measurement indicates the amount of excess, if any, of the target nucleic acid over the amount of a reference nucleic acid.

In another aspect the nucleic acid samples to be analysed are ligated to oligonucleotides which permit amplification, for example by the polymerase chain reaction, and which suitably further permit attachment to a solid support derivatised with oligonucleotides of complementary sequence. In other words, the tags of the method of the invention may be universal amplification primers, such as universal PCR primers.

Thus in one method target nucleic acids are amplified before detection of any label. Following amplification, excess primers may be removed by treatment with a single-strand specific nuclease, such as nuclease S1, or by absorption to a solid support derivatised with the complement of the tag.

The amplified nucleic acid of the sample may then be denatured and hybridised with libraries of labelled probes. Detection of the label may use any of the methods as disclosed herein. Capture to the solid support may be through the complement(s) of the amplification primer(s).

In one aspect PCR amplification may be used. In another aspect multiple displacement amplification may be used. As such the tags of the method of the invention may be primers suitable for multiple displacement amplification.

In all the above examples the detection of the amount of multiple different probes may be carried out simultaneously or sequentially.

The methods of the invention are not limited to the use of 2 probes, and three, 4 or more probes or probe sets may be used for detection of multiple different targets.

Probes may be labelled before or after hybridisation to a target nucleic acid, suitably before.

In another aspect of the invention the method comprises the steps of attaching to nucleic acids present in the sample a tag which allows the nucleic acids to be captured to a solid support, after which all of the tagged nucleic acids in the sample are captured onto a solid support using a ligand for the tag. The captured tagged nucleic acid is contacted with a labelled probe for a first nucleic acid target present in the sample and the amount of probe that binds to the target is detected. After elution of the first probe the captured tagged nucleic acid is contacted with a labelled probe for a second nucleic acid target present in the sample and the amount of probe that binds to the target may be detected. The amounts of the two different probes may be compared.

In another aspect of the invention, microfluidic methods may be used to enhance further aspects of the invention. The small dimensions present in microfluidic environments are conducive to rapid hybridisation and can speed up the process of hybridisation of the probe (sets) to the targets, or alternatively or in addition the hybridisation of the tags to a solid support.

Therefore in one aspect the method of the invention is carried out wholly or in part in a microfluidic environment.

In one aspect the probes are releasable attached to micro beads, for example, via streptavidin—DSB-X coupling. When the sample containing the target molecules is contacted with the beads containing the probes, a first hybridisation step occurs between target and probe.

In a second step, the bound probe:target complexes can be released from the first set of beads (e.g., by biotin molecules which selectively displace the low-affinity DSB-X), and the complexes then contacted with a capture moiety to capture the probe target complex, which may comprise a capture agent for the tag. For example, the capture moiety may be a further set of beads or a plate having a capture moiety such as poly-dT molecules that capture the probe:target complexes via a poly-A tail tag.

Therefore the invention also relates to a method for the measurement of the differences in the amounts of 2 or more nucleic acid targets in a sample, the method comprising the steps of attaching to nucleic acids present in the sample
(1) a tag which allows the nucleic acids to be captured to a solid support; and
(2)providing a solid support having a labelled probe or probe set for a first nucleic acid target present in the sample and a labelled probe or probe set for a second nucleic acid target present in the sample;
(3)contacting the tagged nucleic acids with the solid support to allow capture of the first and second nucleic acid targets;
(4) releasing the labeled ligand:target complexes from the first solid support;
(5) contacting the ligand-target complexes with a ligand for the tag, wherein the ligand for the tag is attached to a solid support; and
(6) measurement of the amount of ligand-target complexes via the label.

Suitably the probe is not a single labelled nucleotide.

In one aspect the ligand-target complexes may be released from the first solid support before contact with the solid support attached to the ligand for the tag. In one aspect the contacting of the tagged nucleic acids with the solid support to allow capture of the first and second nucleic acid targets happens at a first location, the ligand-target complexes are released and the contacting of the ligand-target complexes with a ligand for the tag takes place at a second location. In one aspect the released ligand-target complexes flow from the first location to the second location. In one aspect the first and second location are different wells within a microfluidic channel, and the ligand target complex can flow from the first to the second well.

This principle is disclosed in FIG. 20.

In one aspect the sample is suitably allowed to flow over the probe-solid support complex. In one aspect where the probe target complex is then released, it is allowed to flow over the second solid support to effect capture.

Solid supports are suitably microbeads of 1-50 microns diameter, such as 1, 2, 3, 4, 5, 10, 15, 20, 25, 30, 35, 40, 45 or 50 microns in diameter.

In another aspect of the invention the sample may be divided into a first and second aliquot. The first aliquot is probed with a first probe or probe set labelled with first label for a first nucleic acid target present in the sample and with a second probe probe or probe set labelled with a second (different) label for a second nucleic acid target present in the sample. The second aliquot is probed with the first probe labelled with the second label for a first nucleic acid target present in the sample and a second probe labelled with the first label for a second nucleic acid target present in the sample. This may be referred to as a dye swap approach.

This approach can yield 4 intensity values in two pairs, (I_{L1}, R_{L2}) and (I_{L2}, R_{L1}), where chromosome/first target of interest =I, reference chromosome/second target=R, and L1 and L2 are the different labels. From these values, three ratios can be calculated according to FIG. 16 and equation below, and where comparison of the values obtained is used to determine if there is an imbalance in the amount of the two targets.

$$\begin{matrix}{R}_{1} = \frac{{I}_{L 1}}{{R}_{L 2}} & ↘ \\ {R}_{2} = \frac{{R}_{L 1}}{{I}_{L 2}} & ↗\end{matrix} {R}_{3} = \frac{{R}_{1}}{{R}_{2}} = \frac{\frac{{I}_{L 1}}{{R}_{L 2}}}{\frac{{R}_{L 1}}{{I}_{L 2}}}$$

These ratios are predictable for cases where set I and set R are comparable (e.g., where there is no aneuploidy), and R₁=R₂ as well as R₃=1. However, where there is an excess within set I as compared to set R, the result is different and R₁≠R₂ as well as R₃≠1.

In one aspect the data on the quantity of label is collected by scanning an image reflecting the quantity of label—for example, an image of the fluorescence of a fluorescent probe. Suitably the data collection comprises the steps of obtaining a scanned image, integrating pixel values and then either fitting pixel values to an analytical expression and/or fitting pixel values to an empirical model.

The invention further relates to a method for the measurement of the differences in the amounts of 2 or more nucleic acid targets in a sample, the method comprising the steps of attaching to nucleic acids present in the sample:
(1) a tag which allows the nucleic acids to be captured to a solid support; and
(2) a probe or probe set for a first nucleic acid target present in the sample and a probe or probe set for a second nucleic acid target present in the sample,
wherein each probe comprises 2 primer portions, the primer portions differing between the 2 probes or probe sets, and wherein the probe primers portions serve as targets for amplification primers to amplify the first and second probes, wherein the amplification reaction for the first and second probe uses a labelled amplification primer, and wherein the label for amplification of the first and second probe is different such that the product of the amplification of the first and second probe is a differently labelled amplification product.

The amount of each labelled amplification product may be detected and compared, or the difference in the amount of labelled amplification product may be detected directly.

The probes may be DNA, RNA or modified DNA probes as described herein. Where the probe is RNA then a reverse transcription amplification may be carried out.

In this aspect the probe-target complexes may be captured onto a solid support that binds to the tag. This may be an oligo dT bead. The complex may be eluted or digested from the dT beads.

This approach may also be used with the dye swap approach described herein.

This aspect is described in FIG. 21.

The present invention can also be applied to pre-implantation genetic diagnostics (PGD) and pre-implantation genetic screening (PGS). In one embodiment an individual cell or cells are taken from a blastocyst and the genetic material stemming from this cell or cells is analysed for potential genetic abnormalities or imbalances in amounts of nucleic acid in a cell associated with diseases or disorders, such as aneuploidies, using the method of the invention.

The sample of the invention may therefore be nucleic acid obtained from a cell or cells of a blastocyst.

The methods presented in this invention can be applied to the study of aneuploidies or other imbalances in amounts or nucleic acid in a cell, such as in a single cell, with or without amplification of the genetic material.

In one aspect of the invention the methods are used to detect the presence of circulating fetal nucleic acid derived from the blood of a pregnant woman.

Suitably at least 2 different probes are used, each labelled with a fluorescent label. The total DNA from a sample is then analysed using both probes to detect the relative amounts of, or the difference between each nucleic acid.

Where one target is a chromosome potentially able to be present in three copies and the other target is a chromosome that cannot be present in three copies then the difference between the amounts of the 2 chromosomes can be diagnostic for trisomy.

Thus the invention also relates to the use of the method of the invention in the diagnosis of genetic disorders in a fetus where the fetal DNA differs from the maternal DNA, for example in amount—as in trisomy—or in sequence. The methods may be used for identification of a woman carrying a fetus with aneuploidy, for example.

In one aspect the methods are used to detect possible mutations associated with disease, such as cancer in an individual being screened or assessed for the presence and/ progression of the disease, eg cancer. For example, where a disease is associated with a nucleic acid duplication or deletion, the amount of the nucleic acid at the possible duplication or deletion site can be compared with a suitable control present in a single or known copy per genome to determine if a chromosomal location associated with disease, eg cancer is present.

In a yet further aspect of the invention nucleic acid e.g. DNA obtained from a sample may be amplified, for example by PCR amplification, before any tag is added.

Also claimed herein are kits for use in the methods of the invention.

Kits may comprise any two or more of:
1 A first probe, or a set of first probes, specific for a first genomic target;
2 A second probe, or a set of second probes, specific for a second genomic target;
3 A tag suitable for attachment to a population of nucleic acids irrespective of sequence;
4 An enzyme, or enzymatic system comprising an enzyme and substrate, suitable for attachment of a tag to a population of nucleic acids irrespective of sequence;
5 A label for the first probe and/or second probe;

6 A solid support derivatised with a complement of a tag;

7 A third probe, or a set of probes, specific for a third genomic target.

Kits may comprise any 2, 3, 4, 5, 6 or 7 of the above. Specific kits may include, for example:

A kit comprising a probe or probe set for a first nucleic acid and probe or probe set for a second nucleic acid, wherein the first probe or probe set is for a nucleic acid target associated with a disorder and a second probe or probe set is for a nucleic acid target not associated with the disorder, wherein the disorder is associated with a change in the amount of the first nucleic acid target in a genome.

A kit comprising a tag that may be attached to a nucleic acid to allow that nucleic acids to be captured to a solid support and a probe or probe set for a nucleic acid target associated with a disorder, the disorder being associated with a change in the amount of nucleic acid target in a genome, such as aneuploidy.

It will be understood that particular aspects and embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In one aspect such open ended terms also comprise within their scope a restricted or closed definition, for example such as “consisting essentially of”, or “consisting of”.

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

All documents referred to herein are incorporated by reference to the fullest extent permissible.

Any element of a disclosure is explicitly contemplated in combination with any other element of a disclosure, unless otherwise apparent from the context of the application. The present invention is further described by reference to the following examples, not limiting upon the present invention.

### EXAMPLES

The present invention is hereby illustrated with the following non limiting examples, wherein examples 1-5 are illustrated in schematically FIG. 19 and example 6 is illustrated schematically FIG. 21.

### Example 1

In one preferred embodiment, nucleic acid samples to be analysed are tagged with a moiety which permits attachment to a solid support: examples are a homopolymer of nucleotides added by terminal transferase; an oligonucleotide differing in sequence from any sequence that is known or expected to be present in the target nucleic acids; biotin.

Two or more sets of probes, which comprise nucleic acids complementary to targets whose amounts are to be compared are labelled with labels which allow the amounts of each set to be measured independently of the other(s). For example, the probes may comprise sets of synthetic oligonucleotides, or cloned nucleic acids with sequences complementary to those of the target nucleic acids. The preferred labels attached to the probes comprise fluorescent labels with distinguishable emission spectra.

The target nucleic acids and the sets of labelled probes are mixed in solution and allowed to hybridise. The mixture is then applied to a solid support derivatised with a moiety which will capture the capture tag attached to the target nucleic acids, under conditions which retain the duplexes formed between the targets and the labelled probes. The solid support may comprise a sheet material, for example of glass or plastic, derivatised with a homopolymer complementary to a homopolymer capture tag, or an oligonucleotide complementary to an oligonucleotide capture tag, or streptavidin to capture a biotin capture tag. After washing away non-captured label, the area of sheet material to which the mixture was applied is then analysed in a fluorescence scanner to measure the fluorescence of the two or more fluorophores used to label the probes. In practice, the measurements will have been calibrated with a mixture of nucleic acids of known content of the two or more target nucleic acids.

Alternatively, the solid support may comprise a particulate material, such as a column packing material, or magnetic beads, derivatised with reagents complementary to the tags. The complex formed, as above, between the target nucleic acids and the labelled probes is passed through a column of the column packing material, or is mixed with the magnetic beads, which are then washed to remove non-hybridised tags. Bound probes are then eluted under conditions which remove them from the column or the beads and the fluorescence spectrum of the eluate is measured to quantify the relative amounts of different probes as a measure of the relative amounts of the target nucleic acids in the sample.

Previous inventions measure the presence of foreign nucleic acids by hybridisaion to a solid support and detection via hybridisation of a labelled probe, in a method termed “sandwich hybridisation”. There are two major differences between this and the invention presented here. In sandwich hybridisation (1) capture is selective and sequence specific (2) capture is mediated by a separate oligonucleotide molecule that is not covalently attached to the target molecule. The oligonucleotide molecule comprises two regions; a sequence-specific target capture region and a homopolymer solid support capture region. It is intended that all captured molecules are also hybridised with a labelled DNA oligonucletide probe for detection

In the method presented here, target molecules are suitably modified via covalent attachment of a tag that is intended for non-selective capture of all sequences. Labelling of the captured molecules is sequence specific and selective.

### Example 2

In a second preferred embodiment, as in example 1, nucleic acid samples to be analysed are tagged with a moiety which permits attachment to a solid support: examples are a homopolymer of nucleotides added by terminal transferase; an oligonucleotide differing in sequence from any sequence that is known or expected to be present in the target nucleic acids; biotin.

Labelling reagents comprise two or more sets of probes, which comprise oligonucleotides with one section of sequence complementary to targets whose amounts are to be compared, and a second section that is common to all members of a set representing a region(s) of each target in the sample. This second section acts as a second ‘capture tag’ used to discriminate the nucleic acid targets. Each set is labelled with a different label which allows its amount to be distinguished from the other set(s). The sequences of the common sections of the oligonucleotide reagents are chosen such that they would not form stable duplexes with any nucleic acid in the sample. The preferred labels comprise fluorescent labels with distinguishable emission spectra. The target nucleic acids and the sets of labelled probes are mixed in solution and allowed to hybridise. The mixture is then applied to a solid support derivatised with a moiety which will capture the capture tag attached to the target nucleic acids, under conditions which retain the duplexes formed between the targets and the labelled probes. The solid support may comprise a sheet material or, preferably, a particulate material such as column packing material, or magnetic beads derivatised with a homopolymer complementary to a homopolymer capture tag, or an oligonucleotide complementary to an oligonucleotide capture tag, or streptavidin to capture a biotin capture tag. After washing away non-captured oligonucleotide reagents, the complex formed, as above, between the target nucleic acids and the labelled oligonucleotide reagents is eluted under conditions which remove them from the column.

The relative amounts of the labelled oligonucleotide reagents are determined by flowing them over a lawn of oligonucleotide capture agents attached to a solid support. The oligonucleotide capture agents comprise a mixture of oligonucleotides with sequences complementary to the sequences of each of the capture sections of the labelled oligonucleotide reagents. Conditions are chosen such that the labelled oligonucleotide reagents are initially in molar excess over their complements in the lawn, so that as the mixture flows over the lawn, the labelled oligonucleotide reagents saturate their complements on the surface, until they have been depleted to a level such that they are not in molar excess over their complements. The point at which depletion below excess occurs depends on the concentration of the target in the sample—those in smallest amount are depleted first. The relative amounts of component targets in the sample is measured by measuring the distance migrated of each of the fluorescent labels along the flow path. In practice, the measurements will have been calibrated with a mixture of nucleic acids of known content of the two or more target nucleic acids.

In an alternative embodiment, the lawn of oligonucleotide capture agents is replaced by column packing material derivatised with the mixture of oligonucleotide capture agents. The eluate from the solid support used to capture the duplexes formed between the between the targets and the labelled probes is applied to the column of mixed oligonucleotide capture agents under conditions which allow duplex formation between the common sequence sections of the target-specific sets and their complements on the solid support. Conditions are chosen such that the labelled oligonucleotide reagents are initially in molar excess over their complements in the column, so that as the mixture flows through the column, the labelled oligonucleotide reagents saturate their complements on the support, until they have been depleted to a level such that they are not in molar excess over their complements. The point at which depletion below excess occurs depends on the concentration of the target in the sample—those in smallest amount are depleted first. The relative amounts of component targets in the sample is measured by eluting the column under conditions which dissociate the labelled reagents from the column. The relative amounts of the components in the target are measured from the relative amounts of the corresponding fluorophore measured as the outflow from the column passes a fluorescence detector.

### Example 3

In a third preferred embodiment, as in example 1, nucleic acid samples to be analysed are tagged with a moiety which permits attachment to a solid support: examples are a homopolymer of nucleotides added by terminal transferase; an oligonucleotide differing in sequence from any sequence that is known or expected to be present in the target nucleic acids; biotin.

Labelling reagents comprise two or more sets of probes, which comprise oligonucleotides with one section of sequence complementary to targets whose amounts are to be compared, and a second section that is common to all members of a set representing a region(s) of each target in the sample. This second section acts as a modulator of mobility during electrophoresis, for example capillary electrophoresis, used to discriminate the probes and hence allow quantitation of their corresponding nucleic acid targets. Differential mobility may be conferred by oligonucleotide sections of different length or other moieties which confer different charge or different bulk. Each set is labelled. Labelling may be with a single fluorophore, or sets may be labelled with different labels which allows its amount to be distinguished from the other set(s). The preferred labels comprise the fluorescent labels used routinely in capillary sequencing.

The target nucleic acids and the sets of labelled probes are mixed in solution and allowed to hybridise. The mixture is then applied to a solid support derivatised with a moiety which will capture the capture tag attached to the target nucleic acids, under conditions which retain the duplexes formed between the targets and the labelled probes. The solid support may comprise a sheet material or, preferably, a particulate material such as column packing material, or magnetic beads derivatised with a homopolymer complementary to a homopolymer capture tag, or an oligonucleotide complementary to an oligonucleotide capture tag, or streptavidin to capture a biotin capture tag. After washing away non-captured oligonucleotide reagents, the complex formed, as above, between the target nucleic acids and the labelled oligonucleotide reagents is eluted under conditions which remove them from the column.

The relative amounts of the labelled oligonucleotide reagents are determined by electrophoretic separation, for example, capillary electrophoresis.

### Example 4

In a fourth embodiment, as in example 1, nucleic acid samples to be analysed are tagged with a moiety which permits attachment to a solid support: examples are a homopolymer of nucleotides added by terminal transferase; an oligonucleotide differing in sequence from any sequence that is known or expected to be present in the target nucleic acids; biotin.

Labelling reagents comprise a set(s) of paired probes, which comprise oligonucleotides with one section of sequence complementary to targets whose amounts are to be compared, and a second section that is common to all members of one of the pair of a set representing a region(s) of a target in the sample. The probes specific for the second member of the paired targets contain a second section which is complementary in sequence to the first member of the pair, which is expected to be in excess of or equivalent in amount to the second member of the pair of targets. The members of the first paired set are labelled with a fluorophore at one end of the second section of the tag. The members of the second paired set are labelled with a quencher to the fluorophore at the other end of the second section of the tag.

The sequences of the common sections of the oligonucleotide reagents are chosen such that they would not form stable duplexes with any nucleic acid in the sample.

The target nucleic acids and the sets of labelled probes are mixed in solution and allowed to hybridise under conditions which allow duplex formation between the target specific sections of the tagged oligonucleotides, but not between the common sections of the labelled probes and their complements in the second members sets of the paired probes. The mixture is then applied to a solid support derivatised with a moiety which will capture the capture tag attached to the target nucleic acids, under conditions which retain the duplexes formed between the targets and the labelled probes. The solid support may comprise a sheet material or, preferably, a column packing material, derivatised with a homopolymer complementary to a homopolymer capture tag, or an oligonucleotide complementary to an oligonucleotide capture tag, or streptavidin to capture a biotin capture tag. After washing away non-captured oligonucleotide reagents, the complex formed, as above, between the target nucleic acids and the labelled oligonucleotide reagents is eluted under conditions which remove them from the column. The complementary sections of the labelled tagged oligonucleotides in the eluate are allowed to hybridise, bringing the quencher and fluorophore into juxtaposition. A fluorescence measurement indicates the amount of excess, if any, of the target nucleic acid over the amount of a reference nucleic acid.

In practice, the measurements will have been calibrated with a mixture of nucleic acids of known content of the two target nucleic acids.

### Example 5

In a fifth embodiment, nucleic acid samples to be analysed are ligated to oligonucleotides which permit amplification by the polymerase chain reaction and which further permit attachment to a solid support derivatised with oligonucleotides of complementary sequence.

Following amplification, excess primers are removed by treatment with a single-strand specific nuclease, such as nuclease S1, or by absorption to a solid support derivatised with their complements.

The tagged PCR products are then denatured and hybridised with libraries of labelled probes, as in Examples herein. The following steps, which permit the measurement of the differences in labels associated with the two or more targets to be analysed follow the corresponding methods described for Examples herein, except that the capture to solid support is through the complement(s) of the PCR primer(s).

### Example 6

In a sixth example, as in example 1, nucleic acid samples to be analysed are tagged with a moiety which permits attachment to a solid support: examples are a homopolymer of nucleotides added by terminal transferase; an oligonucleotide differing in sequence from any sequence that is known or expected to be present in the target nucleic acids; biotin

Two or more sets of probes, which comprise nucleic acids complementary to targets whose amounts are to be compared are used. These sets of probes comprise mainly target complementary sequence but have a non-complementary unique sequence at each end, that is to be used as target specific amplification primer. The target nucleic acids and the sets of probes are mixed in solution and allowed to hybridise.

The probe:target tagged hybrid nucleic acid sample as well as the tagged target only are captured to a solid support. The single stranded free probe is washed away. The probe can be removed from the solid support by either elution or digestion of its complementary strand.

The eluted probe is then amplified using target specific labelled primers. For example, chromosome 21 forward and reverse target primer set would be labelled with dye1 and a reference chromosome target forward and reverse primer set would be labelled with dye 2

Following amplification, excess primers are removed by treatment with a single-strand specific nuclease, such as nuclease S1, or by absorption to a solid support derivatised with their complements.

The labelled PCR products are then denatured and hybridised to a solid support that comprises a mixture of the target specific primer complements for quantification.

### Example 7

### General Protocol

It is anticipated that the following general protocol will be generally useful in the present invention, eg in NIPD of eg fetal aneuploidy through chromosome specific detection.
Label target and reference chromosome specific probes e.g. paint probes with different dyes
Purify DNA fragments from whole blood and tag with a known sequence (eg addition of a Poly A tail)
Hybridise DNA fragments to labelled paint probes from target and reference chromosomes
Capture all DNA fragments to a solid support comprising a tag complement eg an oligo dTn sequence. (Only target and reference fragments will be labelled)
Scan solid support on scanner
Quantitate amount of target and reference fragments by calculating the total integrated intensity of a feature
A higher value of the test: reference sample ratio for the target chromosome relative to the reference chromosome might be indicative of a disorder, eg a trisomy.

### Example 8.1

### Model to Show the Detection of a Difference in Hybridisation Signal Between a Maternal Cell Free (“cf”) DNA Sample Containing a Disomy or Trisomy 21 Fetus

To validate the approach of the invention a model system was designed wherein samples of known numbers of labelled target molecules were captured to a solid support.

Samples were generated to reflect the following:
The number of molecules of “maternal chromosome 21 cf DNA” in 10 mls blood
The number of molecules of “maternal+normal fetal chromosome 21 cf DNA” in 10 mls blood with 4-10% total fetal content
Numbers of molecules of “maternal+trisomy fetal chromosome 21 cf DNA” in 10 mls blood with 4-6% total fetal content

The start point was to capture labelled target from a solution containing a concentration that represents the number of cf DNA molecules found in 10 mls maternal blood.

Further concentrations of nucleic acid were then added that represent the increases found as a result of a either disomy or trisomy 21 fetus in early pregnancy.

The samples were hybridised in quadruplicate for replicate analysis.

In model system 1

The Target was Hba1 IVT
Length: 600 bases
Degree of labelling :12 fluorophores per molecule
Probe: chimeric capture probe
Length: 70 bases; 50 dT+20 GS

Features of model system 1
Robust system that reproducibly yields>90% combined pick up and detection efficiency
Optimised capture probe
Highly labelled pure target molecule

The slide was scanned on a conventional microarray scanner (Agilent, 5 um resolution)

The samples were quantified (using GenePix 6.0) by calculating the total integrated signal intensity of each feature.

The results were analysed to determine if there was a significant difference between the model Disomy and Trisomy samples.

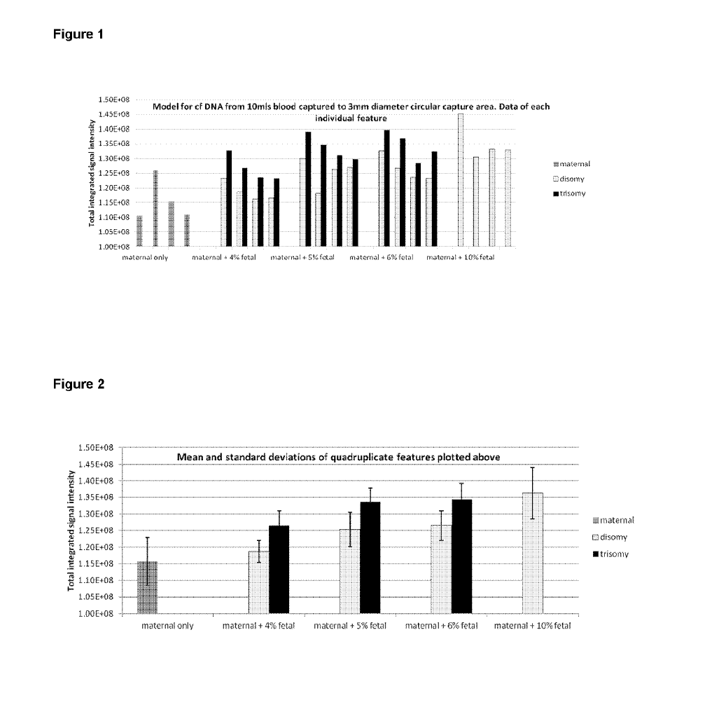

Data is given in FIGS. 1 and 2.

### Conclusions

The results on this model system indicate that detection of small changes in DNA concentration such as those found between fetal disomy and trisomy are detectable by ratios generated in a single colour system on a low resolution scanner
The variation between the replicates is derived mainly from two sources:
   Variation in the local background
   Contribution of pixel outliers
These two sources of variation can be removed by
   A two colour system whereby the test and reference samples are co-located
   Identification and exclusion of the pixel outliers from the data analysis

### Example 8.2

### Model for Chromosome 21 Detection Using Single Colour RNA Probes

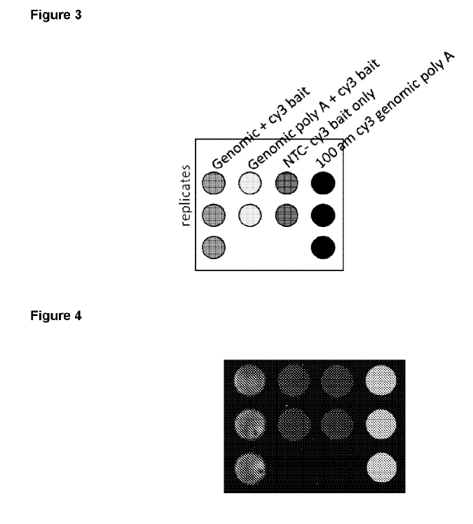

The previous Example 8.1 showed detection of a labelled target IVT used as a model for cf DNA. The concentration of the target represented the calculated average number of chromosome 21 specific molecules in 10 mls maternal blood. In these experiments, model system 2, the cf DNA is represented by sheared genomic DNA. The sheared DNA was tagged with poly dA, denatured, labelled via hybridisation of labelled RNA probes and captured to a dT lawn.
The DNA is sheared under conditions that generate a distribution of fragments with a median length of 160-180 base pairs
The sheared DNA was purified using the Agencourt PCR purification kit as described by the manufacturer. The DNA fragments were poly A tailed using terminal transferase as described by the manufacturer
A library of 120 base RNA probes and comparable genome coverage as chromosome 21 (50 Mb;1% of the genome) was used as a model test system. The RNA probes were labelled and purified with cy dye using the ULS Kreatech labelling kit according to the manufacturer's instructions. Average label density: 3 fluors/molecule
Equimolar concentrations of tailed DNA fragments and library were mixed such that the RNA probes were in 100 fold excess of their target complements. The mixture was heated to denature the double-stranded target and allowed to hybridise at 65° C. for 20 hours
After incubation samples were pipetted directly into wells and overlaid with mineral oil. Capture to dT₇₀ lawn was for 1 hr at 40° C. The capture slide was washed and scanned on a conventional scanner.
Data is given in FIGS. 3-5.

### Conclusions

The results indicate that a detectable signal is achieved on a conventional scanner from a sample representing 10mIs cf DNA, by capture of tagged target molecules following hybridisation of labelled chromosome 21 specific RNA probes

### Example 8.3

### Dual colour RNA probe model experiments and detection of trisomy samples

Modelled with 10% fetal content

The previous experiment showed scanner detection of an NIPD sample (10 mls) amount sheared genomic DNA with a single colour 50Mb RNA probe library.

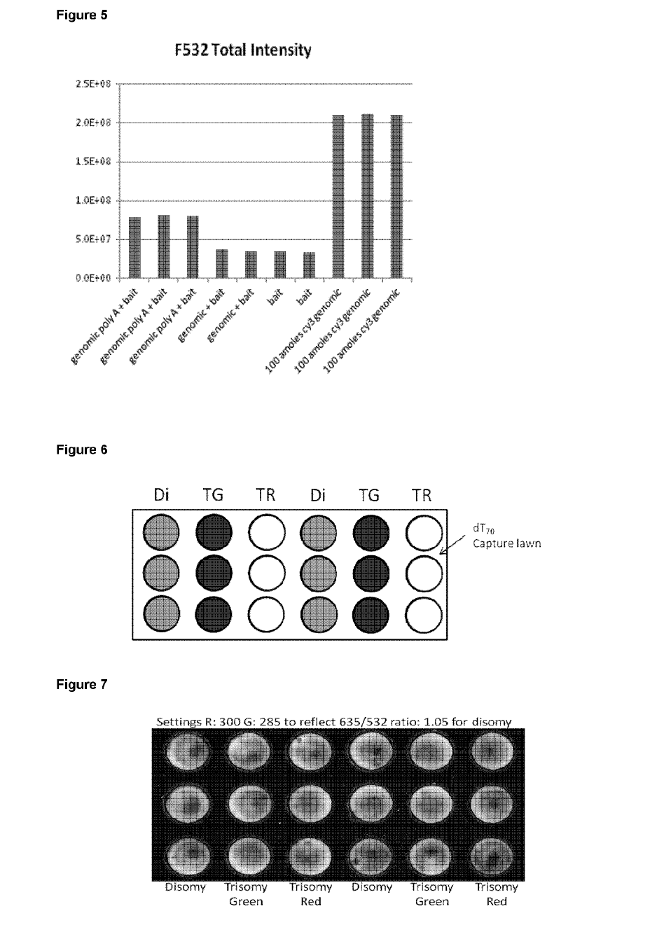

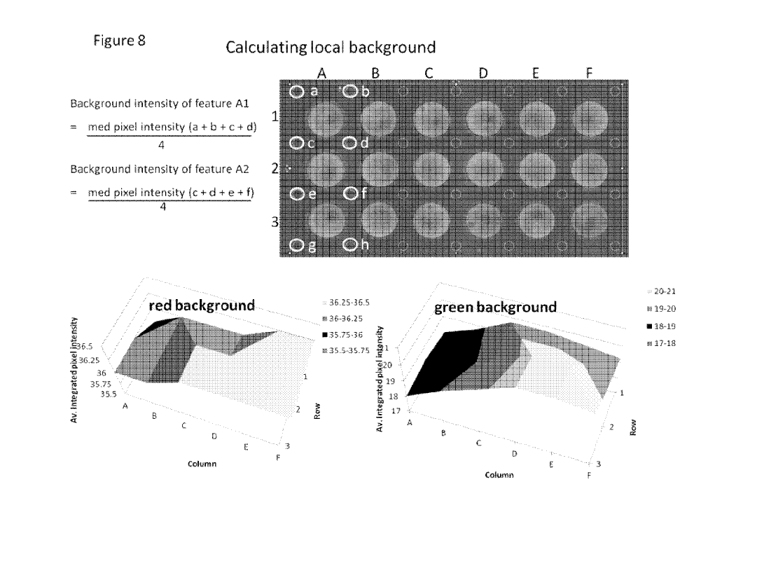

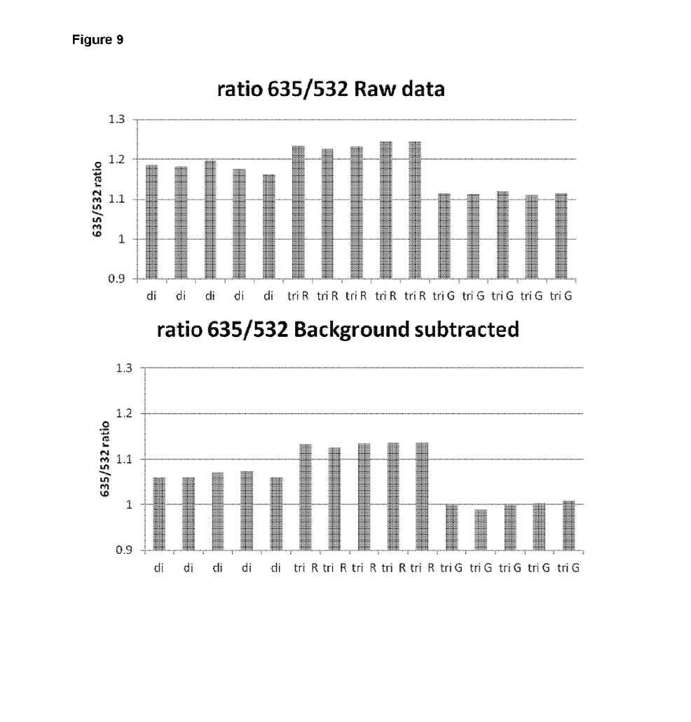

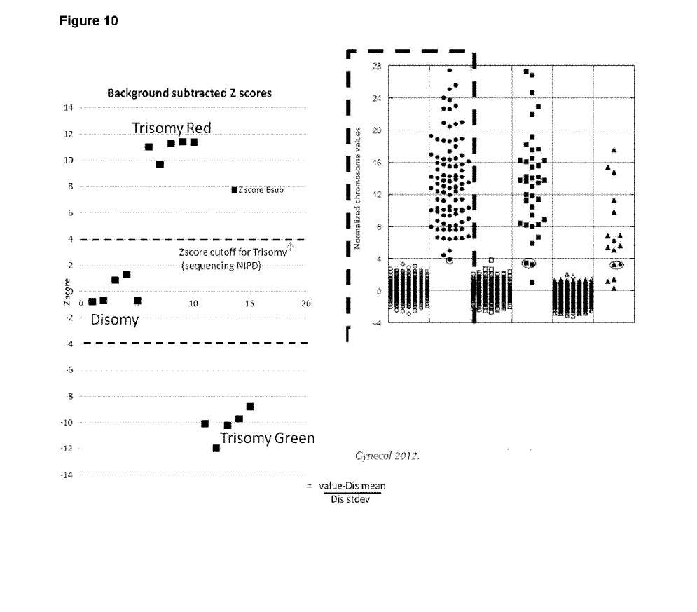

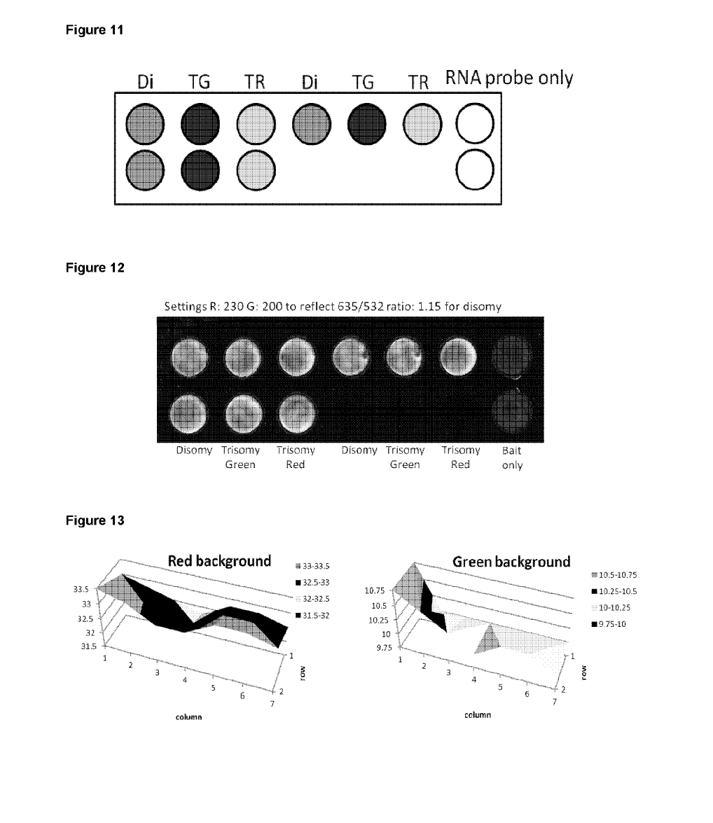

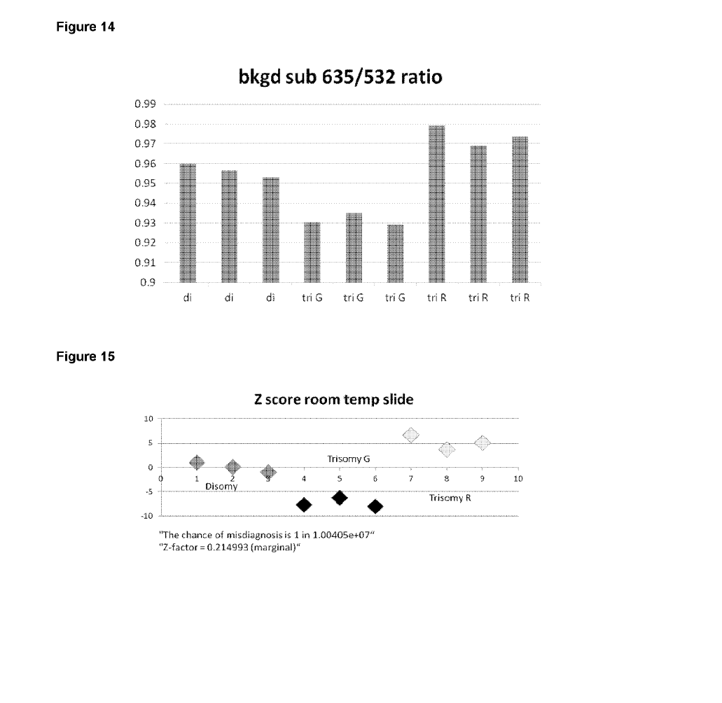

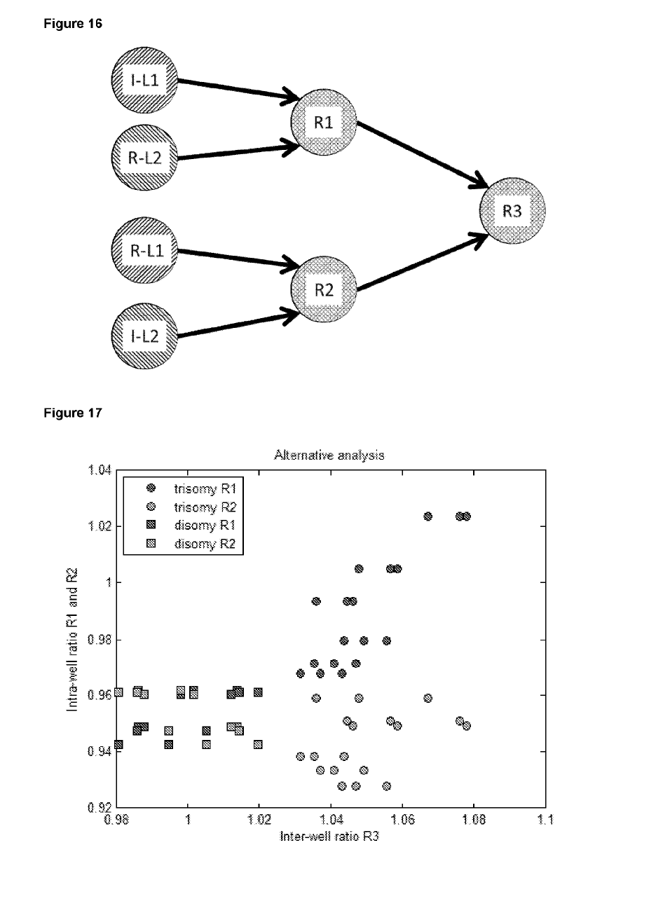

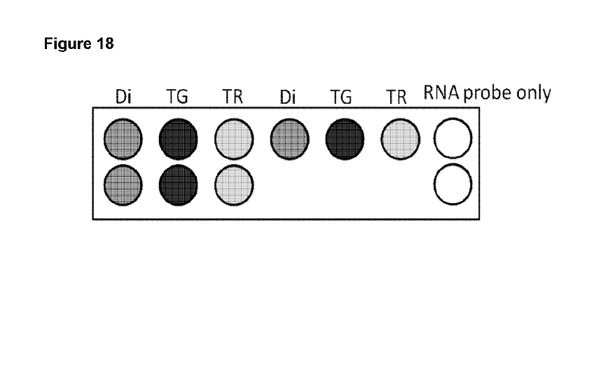

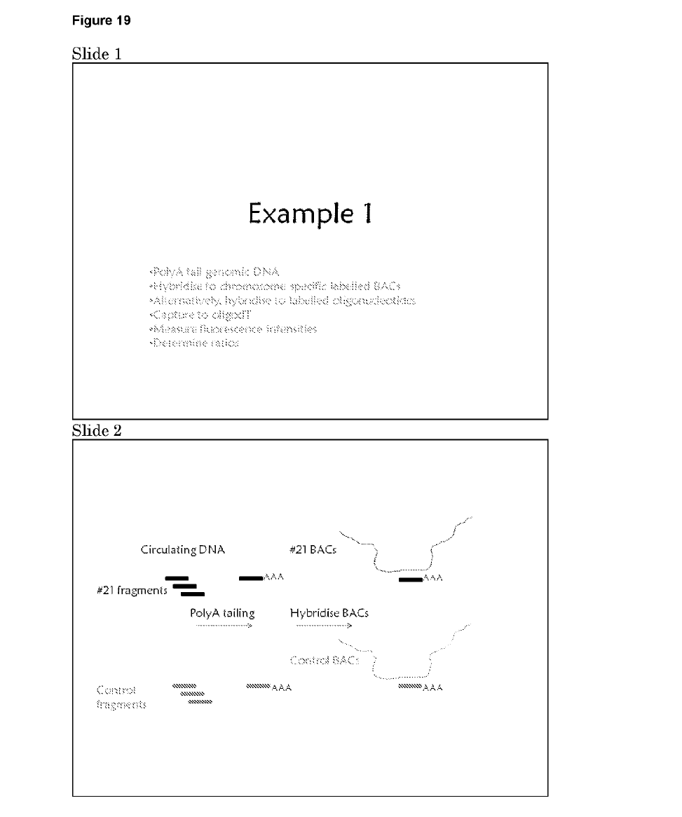

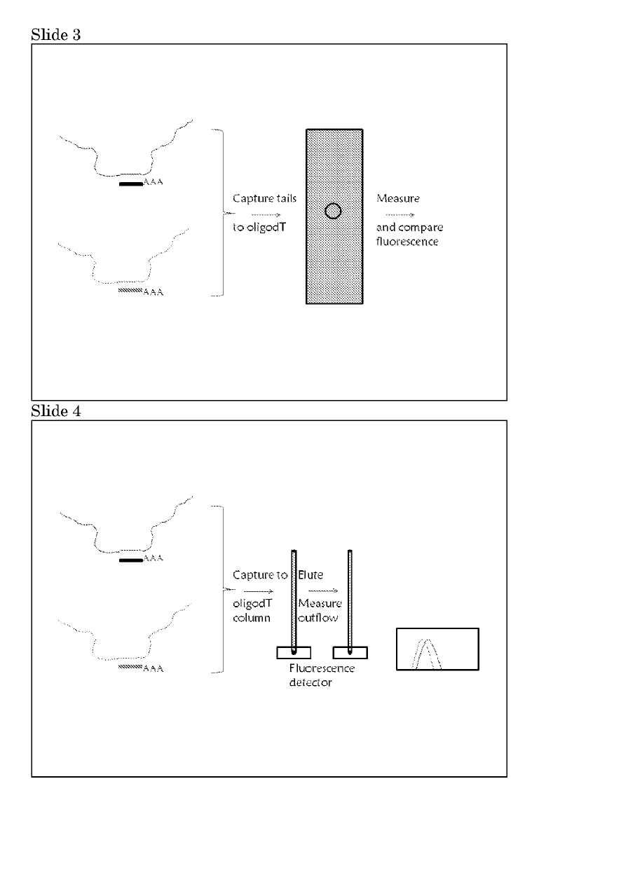

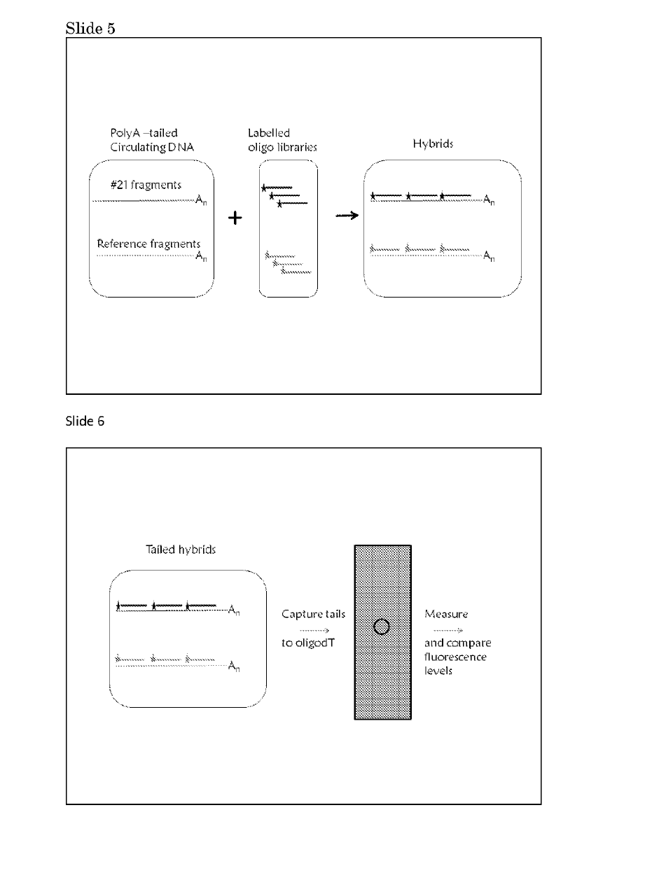

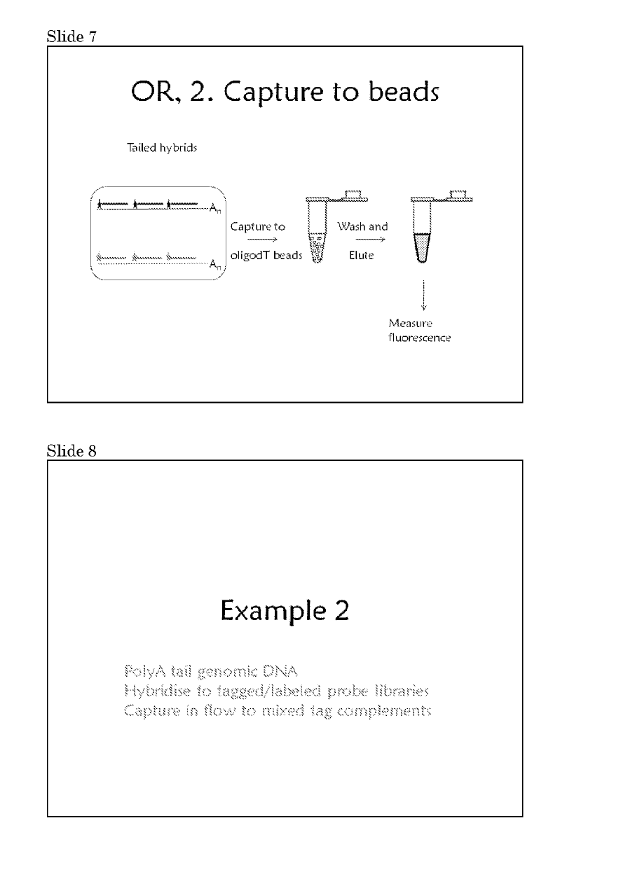

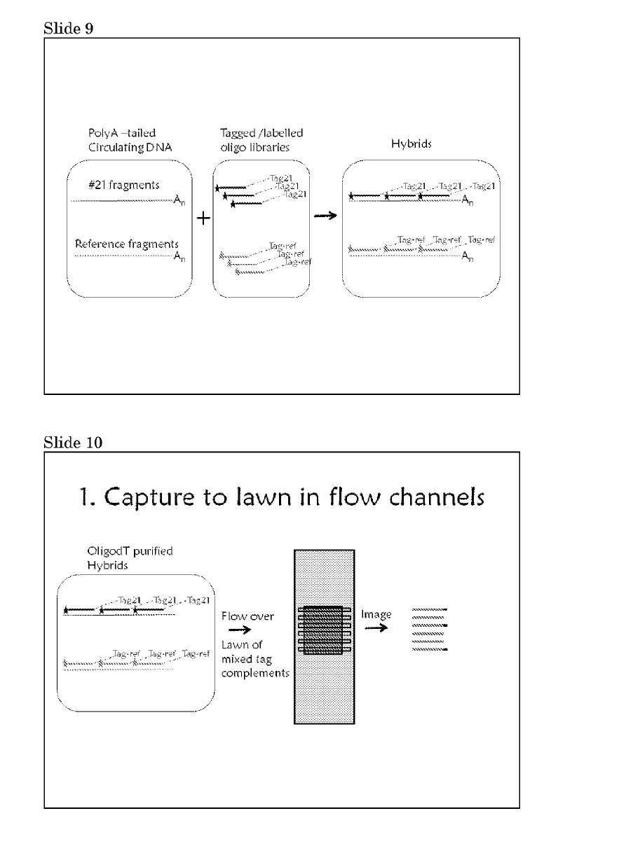

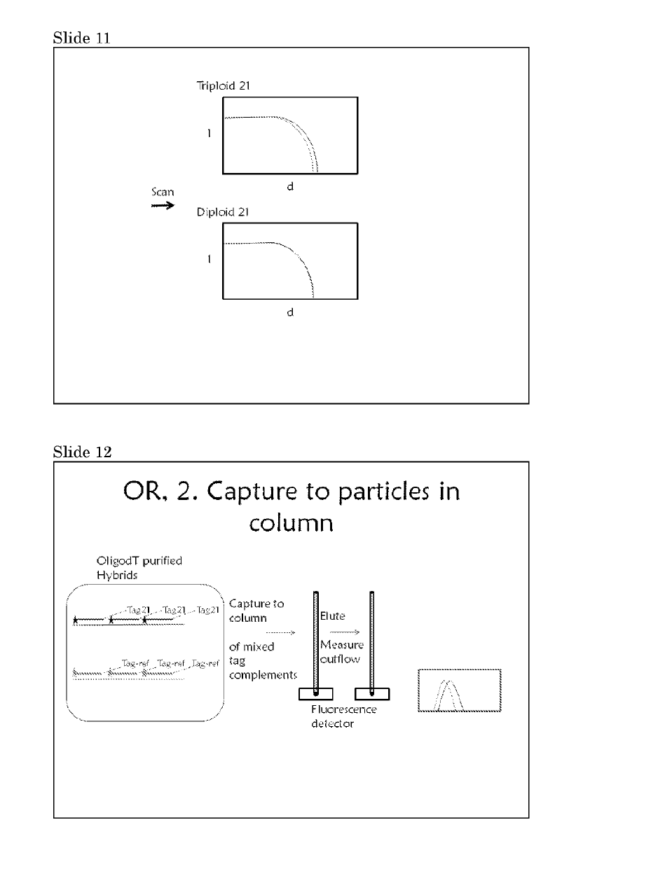

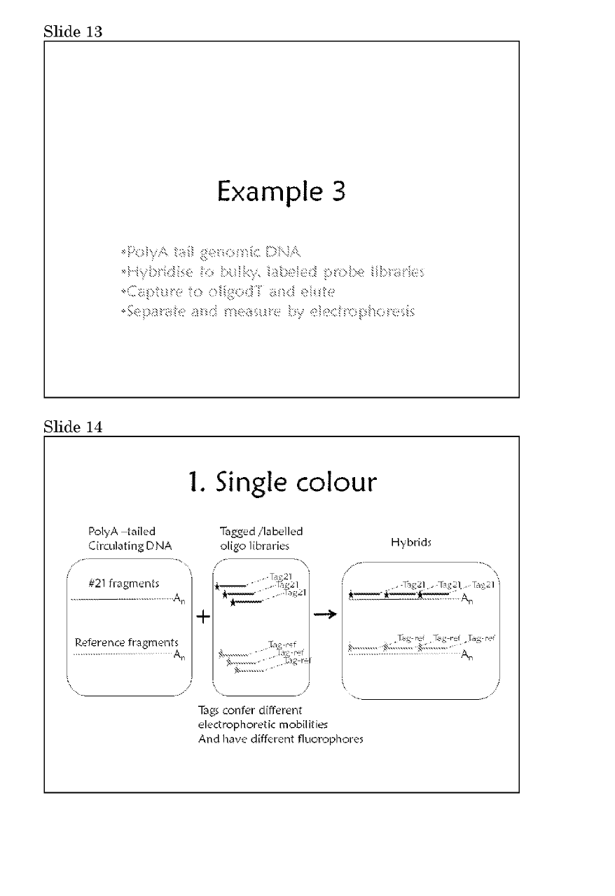

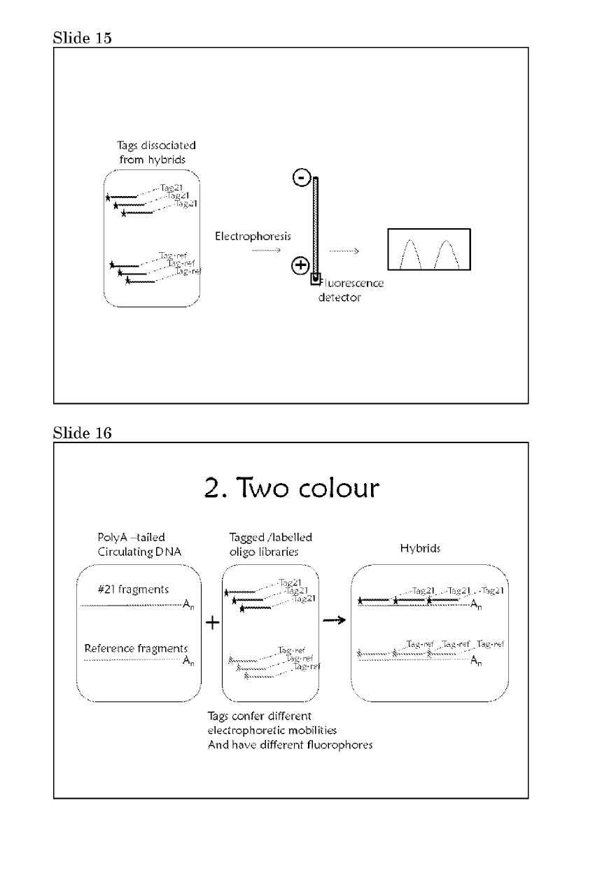

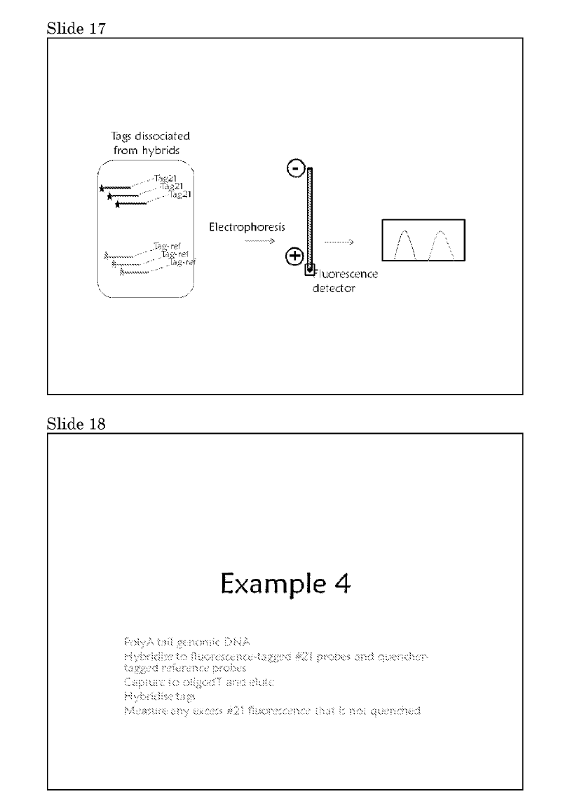

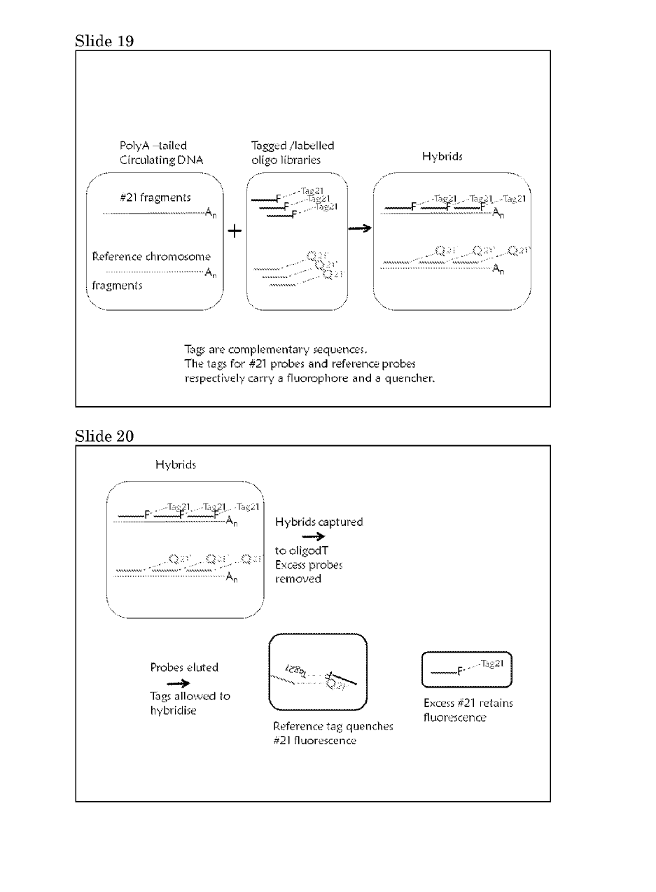

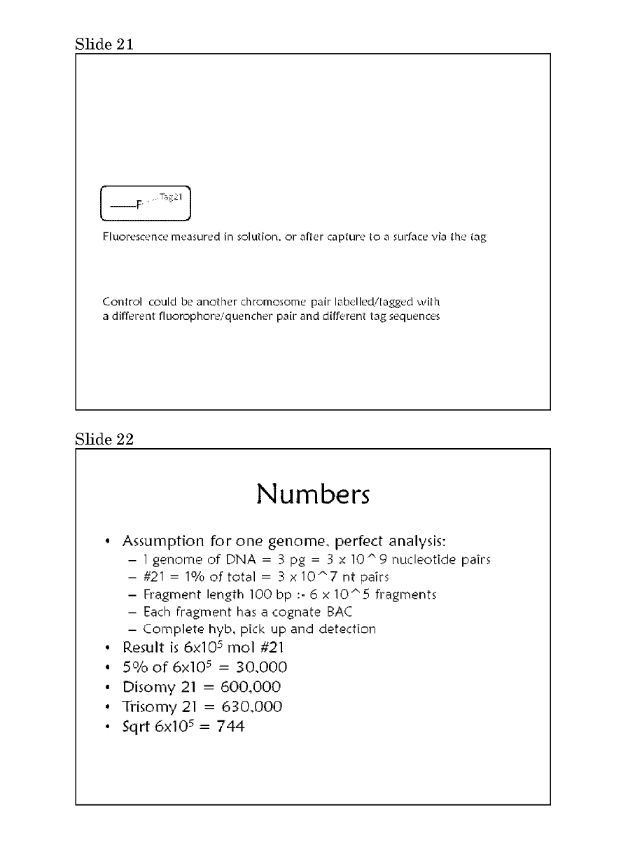

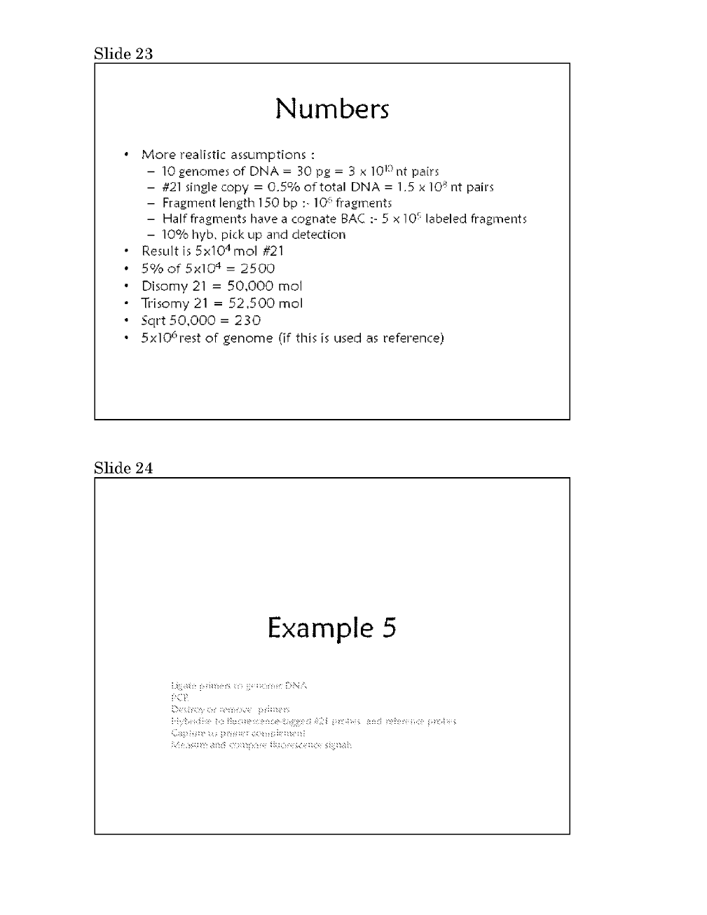

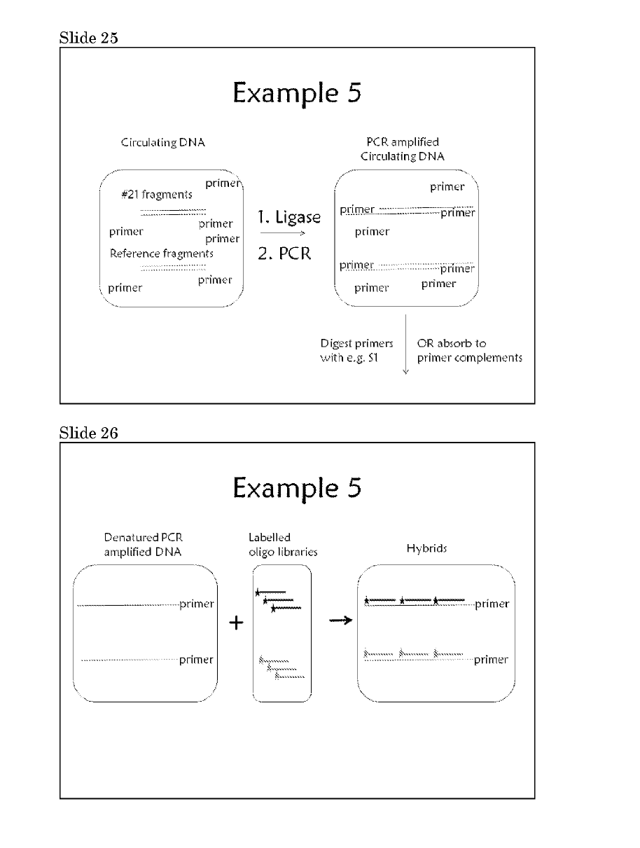

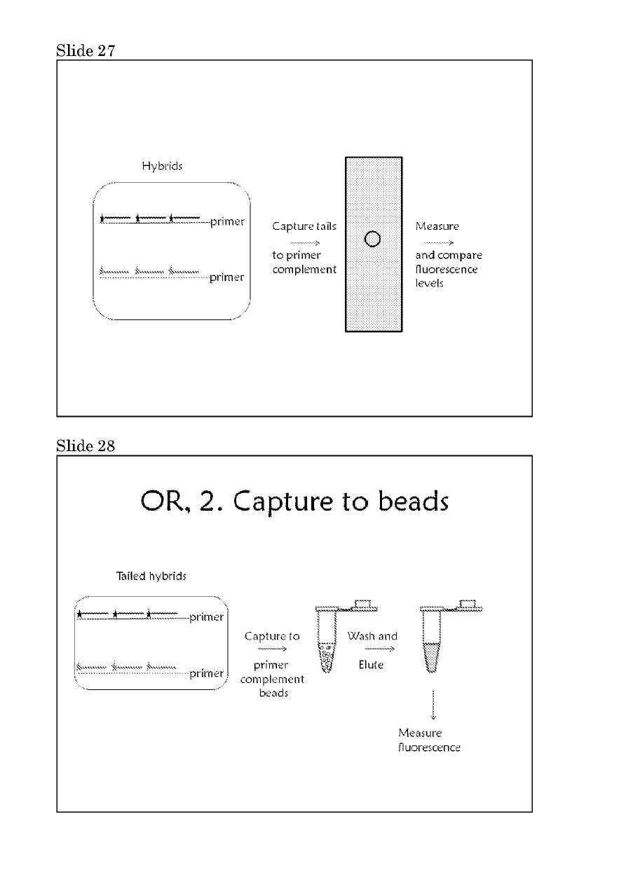

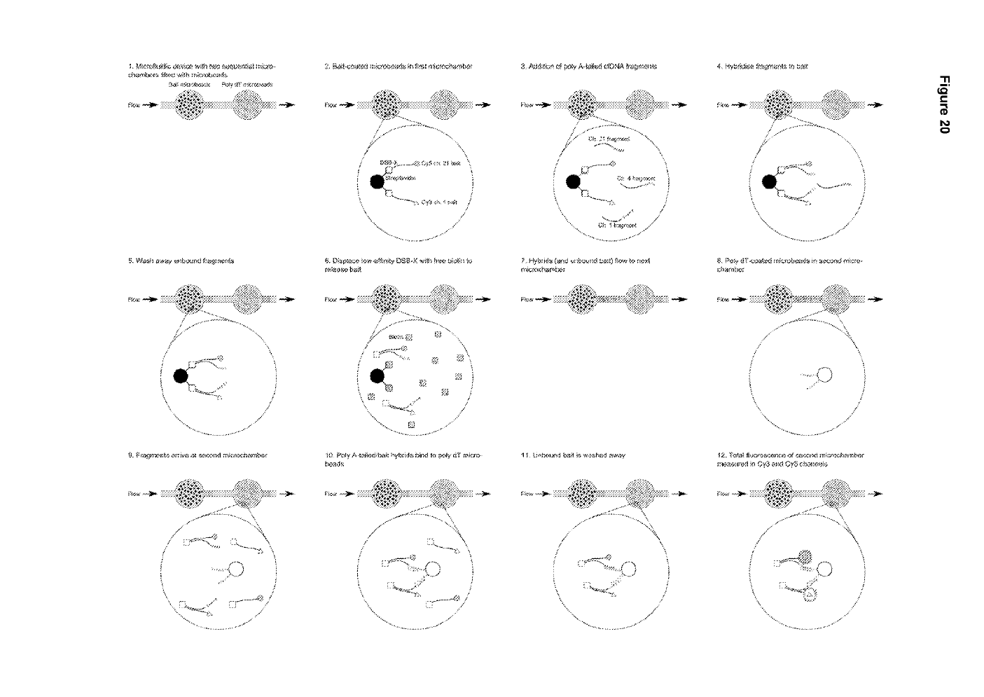

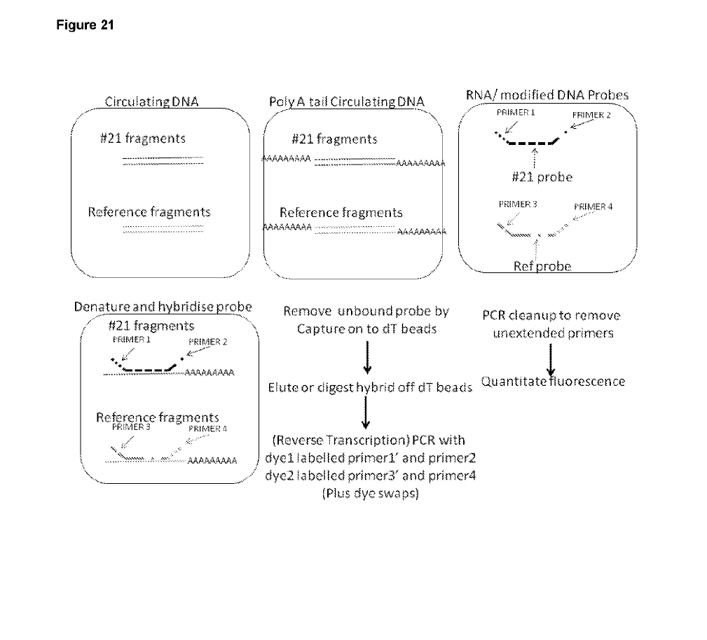

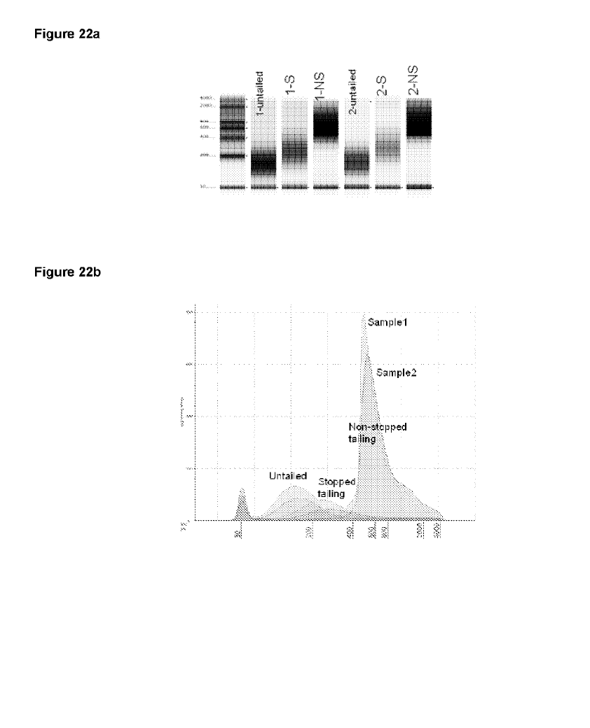

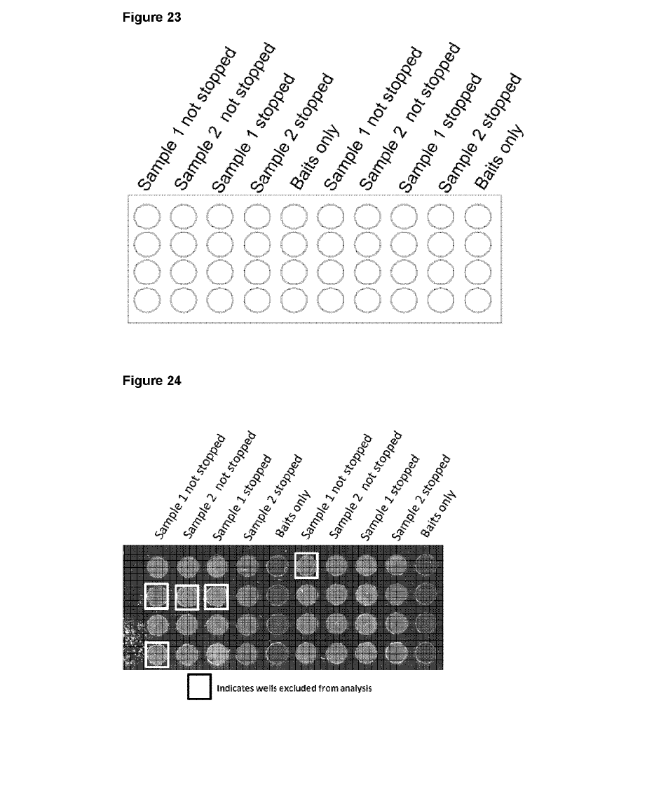

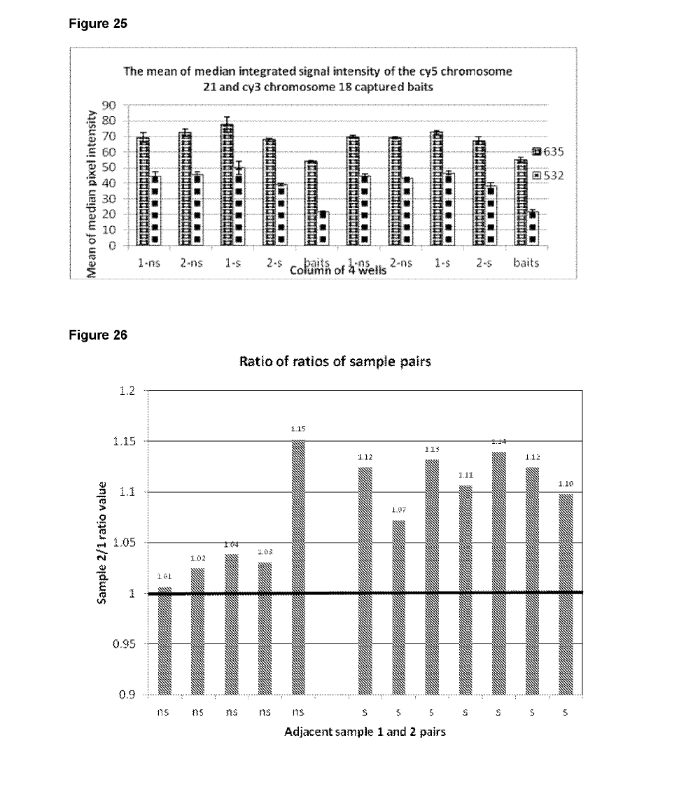

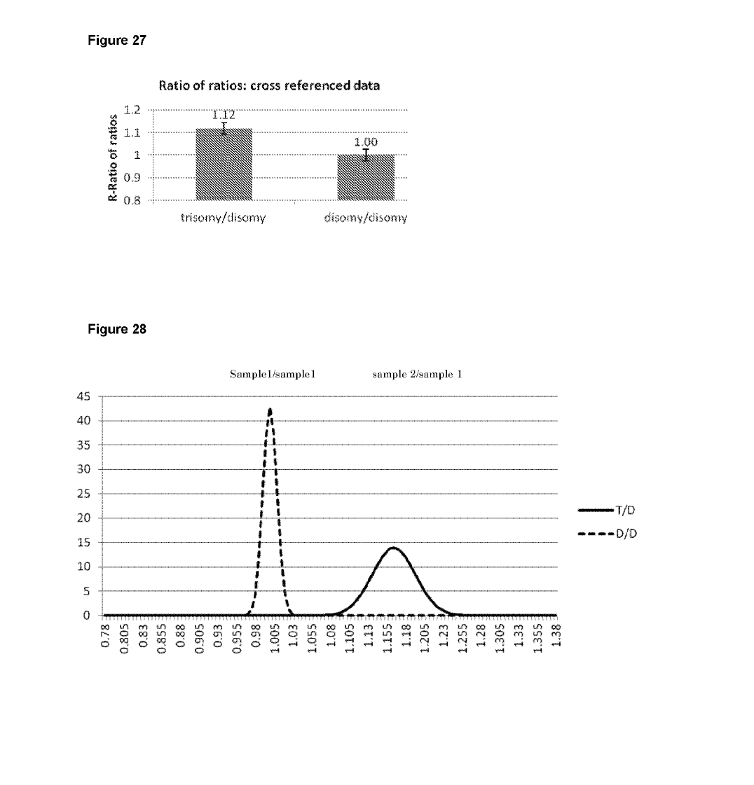

In this experiment the difference between disomy and trisomy is investigated in an NIPD sample amount (10 ng) at 10% fetal content using a two colour RNA probe model system.
Three tubes of master mix were made up
1. mastermix of 40 μl (20 wells) Disomy cy3 and cy5 RNA probes was made up containing
   20×10 ng (75 fmoles) sheared genomic polyA DNA
   20×3 ng (75 fmoles) each of cy3 and cy5 Kreatech labelled RNA probe library (50 MB)
2. A mastermix containing genomic DNA and cy3 RNA probe library to generate cy3 trisomy.
3. A mastermix containing genomic DNA and cy5 RNA probe library to generate cy5 trisomy.
After incubation overnight at 65 degrees, 0.25 ng (5% of 5 ng) mastermix 2 was added to 12 ul (6 samples) mastermix 1 to generate 6 trisomy green samples
Similarly, mastermix 3 was added to mastermix 1 to generate the trisomy red samples.
2 ul samples were loaded into each well and overlaid with 6 ul mineral oil and incubated at room temp (22) for 90 minutes.
Data is given in FIGS. 6-10.

Modelled with 5% fetal content.
The experimental set up was similar to that described in experiment 8.3.
Data is given in FIGS. 11-15.

### Conclusions

The two colour exome model demonstrates detection of fetal trisomy in a representative cf DNA sample of genomic DNA at both modelled fetal concentrations
The signal intensities in (2) represent that likely to be achieved long sample and 5% fetal content
The quality of the data may be improved by optimised analysis e.g see Example 9 and Example 10

### Example 9

### Automated Data Extraction from an Image

Data can be extracted automatically from the resultant images of a scan. One such method is detailed in Listing 1, showing a MATLAB function that identified the features against the background and determines their extent. In the case shown here, discrimination of the features against the median of the pixel intensities of the whole slide works well; in other cases, other criteria may be chosen.

In order to improve background subtraction against artifacts far away from the feature of interest, yet take advantage of the full image to estimate the background, weighted averaging of the background may be useful, giving a higher weight to background closer to individual features. One possible method is outlined in listing 2, which in turn makes use of listing 3.

Listing 1

function [out, cc] = find3mmFeatures(im)
% out = find3mmFeatures(im)
% find the 3mm features in an image represented in the matrix im; im is
% assumed to stem from imread and could be (most likely) of class uint16.
%
% output is a label matrix of same size, where

| | |
|---|---|
| % | background: 0 |
| % | features: integers ranging from 1..N |

%
% See also: imread, imopen, imclose, strel, medfilt2
% minArea: pixel count of the features is larger than this value
minArea = 150000;
% Eccentricity (0 for ideally round, 1 for line): empirically determined
% maximum acceptable eccentricity
maxEcc = 0.5;
% smooth the image and get rid of “small stuff”
J = medfilt2(im, [3 3]);
% the features stand out against the median of the slide; sometimes, this
% does not get rid of some of the noise, particularly towards the edges of
% the image
bw = (J>median(double(im(:))));
% two different structural elements: this reduces the noise better and
% smoothes more in the second step
% in addition, imfill fills in any holes that may be inside features
SE1 = strel(‘disk’, 10);
SE2 = strel(‘disk’, 20);
bw2 = imfill(imclose(imopen(bw, SE1), SE2), ‘holes’);
% find out meta-information about the detected features (bwconncomp) by
% using regionprops; useful for discrimination are the Area and the
% Eccentricity (0 for ideally round, 1 for line): empirically
cc = bwconncomp(bw2);
rp = regionprops(cc, {‘Area’, ‘Eccentricity’});
idx = ( ([rp.Area]>minArea) & ([rp.Eccentricity]<maxEcc) );
% eliminate the features that do not fall under this criterion
cc.PixelldxList(~idx) = [ ];
cc.NumObjects = numel(cc.PixelldxList);
% the next command creates a matrix of the same size as the image, with the
% feature marked with the numbers of the indices in rp.
out = labelmatrix(cc);

Listing 2

function wbg = weightedBackgroundForFeature(R, G, L, rp, feature)
% wbg = weightedBackgroundForFeature(R, G, L, f)
% the mask for the background shall always exclude the features, and an
% area around the features. This is why the mask is set up regardless of
% the feature that is being looked at. The morphological dilation is used
% to expand the features into their adjacent background, which is excluded
% in case that there is some light leakage or non-specific binding
% surrounding the features.
mask = imdilate( (L~=0), strel(‘disk’, 30) );
mask = ~mask;
% now shrink the features a little bit
% L = imerode(L, strel(‘disk’, 30));
% background images
bgr = double(medfilt2(immultiply(R, mask), [5 5]));
bgg = double(medfilt2(immultiply(G, mask), [5 5]));
% setting up the function to calculate the distance from the feature
[X,Y] = meshgrid(1:size(R,2), 1:size(R,1));
wgh = @(xc, yc)( 1./sqrt( (X-xc).\hat{ }2 + (Y-yc).\hat{ }2) );
% calculating the weight matrix
k=feature;
D = wgh(rp(k).Centroid(1), rp(k).Centroid(2));
% return values: the weighted means
wbg.r = wmean(bgr(mask), D(mask));
wbg.g = wmean(bgg(mask), D(mask));

Listing 3

```
function [xbar, wbar] = wmean(x, w)
% [xbar, wbar] = wmean(x, w)
% weighted mean of data x given weights w; if w is not given, then the
% weights are assumed to be 1 for all data, and the resulting mean is
% identical to the normal function mean
%
% Additional information: http://en.wikipedia.org/wiki/Weighted_mean
%
% See also: mean
switch(nargin)
  case 1
    xbar = mean(x);
    wbar = 1;
    return;
  case 2
    % normal case, continue with the normal function below
  otherwise
    error(‘wmean called with wrong number of arguments.’);
end
% sanity check: are the sizes of the arrays x and w identical?
if (size(x) ~= size(w))
  error(‘wmean: sizes of “x” and “w” are not idential’);
end
% ensure that w is of type double
w = double(w);
xbar = sum(double(x).*w)/sum(w);
end
```

### Example 10

### Experimental Design Based on Dye-Swap

### Introduction

The experimental design proposed in the other sections of this patent application is based on the comparison between two differently labelled sets of molecules of interest, for example a set of fragments of a chromosome of interest (set I) and a similar set from a reference chromosome (set R). If there is an excess of molecules in set I compared to set R, then certain conclusions can be drawn, for example the presence of an aneuploidy.

This design relies in some aspects on signal comparison between two different dyes that may or may not have similar absorption coefficients and quantum yields. These differences can either be designed out (e.g., inclusion of more or fewer reference fragments in set R compared to set I), or taken into account during data analysis (e.g., by use of a known normalisation factor).

On the other hand, it is possible to split the initial sample and perform two experiments where set I and set R are labelled using labels L1 and L2, thereby creating sets (I-L1, R-L2) and (I-L2, R-L1). Not only does this enable finding the normalisation factor as typical application in, e.g., microarray experiments, but it can even be used to improve the data analysis and reliability of the experiment.

### Description

By way of example, the experiment can yield 4 intensity values in two pairs, (I_{L1}, R_{L2}) and (I_{L2}, R_{L1}). From these values, three ratios can be calculated according to FIG. 16 and the equation:

$$\begin{matrix}{R}_{1} = \frac{{I}_{L 1}}{{R}_{L 2}} & ↘ \\ {R}_{2} = \frac{{R}_{L 1}}{{I}_{L 2}} & ↗\end{matrix} {R}_{3} = \frac{{R}_{1}}{{R}_{2}} = \frac{\frac{{I}_{L 1}}{{R}_{L 2}}}{\frac{{R}_{L 1}}{{I}_{L 2}}}$$

These ratios are predictable for cases where set I and set R are comparable (e.g., where there is no aneuploidy), and R₁=R₂ as well as R₃=1. However, where there is an excess within set I as compared to set R, the result is different and R₁≠R₂ as well as R₃≠1. The following table illustrates this by simulated example:

| | Disomy | Disomy | Disomy | Trisomy | Trisomy | Trisomy | Trisomy |
|---|---|---|---|---|---|---|---|
| I-L1 | 836 | 798 | 775.2 | 874 | 817 | 782.8 | 771.4 |
| R-L2 | 880 | 840 | 816 | 880 | 840 | 816 | 808 |
| I-L2 | 836 | 798 | 775.2 | 836 | 798 | 775.2 | 767.6 |
| R-L1 | 880 | 840 | 816 | 920 | 860 | 824 | 812 |
| R1 | 0.95 | 0.95 | 0.95 | 0.993182 | 0.972619 | 0.959314 | 0.954703 |
| R2 | 0.95 | 0.95 | 0.95 | 0.908696 | 0.927907 | 0.940777 | 0.94532 |
| R3 | 1 | 1 | 1 | 1.092975 | 1.048186 | 1.019704 | 1.009925 |
| set I | 220 | 210 | 204 | 230 | 215 | 206 | 203 |
| set R | 220 | 210 | 204 | 220 | 210 | 204 | 202 |
| L1 factor | 3.8 | 3.8 | 3.8 | 3.8 | 3.8 | 3.8 | 3.8 |
| L2 factor | 4 | 4 | 4 | 4 | 4 | 4 | 4 |
| foetal fraction | 0.1 | 0.05 | 0.02 | 0.1 | 0.05 | 0.02 | 0.01 |

For this table it has been assumed that set I and set R contain the same number of fragments, and that labels L1 and L2 yield different signals per fragment (L1/L2 factors).

### Experimental Results

A simulated fetal fraction of 5% was added to genomic DNA (as per Example 8), disomy and trisomy were simulated in the same way. The following ratios are expected based on the observed signal intensities for Cy3 and Cy5 channels:

| | Expected | Measured |
|---|---|---|
| | value | value |
| R1 | Same as R2 | 0.954 ± 0.008 |
| (disomy) | | |
| R2 | Same as R1 | 0.954 ± 0.008 |
| (disomy) | | |
| R3 | 1.00 | 1.000 ± 0.013 |
| (disomy) | | |
| R1 | Different from | 0.990 ± 0.020 |
| (trisomy) | R2 | |
| R2 | Different from | 0.943 ± 0.011 |
| (trisomy) | R1 | |
| R3 | 1.05 | 1.050 ± 0.013 |
| (trisomy) | | |

FIG. 17 shows the clear clustering of the data points along the axes R3 along the horizontal axis as a common reference, and R1 and R2 for the two data points per experiment.

Description of the experiment:
Simulated trisomy/disomy data based on
   Genomic DNA
   2 RNA probe libraries, one labelled with Cy3, the other one with Cy5
   Mixing according to
      Disomy—7 ul cy3 mix+7 ul cy5 mix+0.18 ul×buffer
      Trisomy green—7.18 ul cy3 mix+7 ul cy5 mix
      Trisomy red—7 ul cy3 mix+7.18 ul cy5 mix
Layout of the slide is according to the FIG. 18
Data analysis combines pairwise Di/Di, and TG/TR results
   Dye swap equations

There are two types of molecules in the experiment that lead to measurable signals. The molecules of interest are called here I, and the reference molecules are called R. There is a fraction ΔI that represents the excess due to a trisomy of the fetus. Noise, such as detector noise and non-specific binding (i.e., increasing the signal) or secondary structure (i.e., reducing the signal), is summarised in the terms δi and δr. The fraction β is by design close to 1, but may not be exactly 1. The two labels L1 and L2 may have different quantum yield and absorption cross section, and this is summarised in the factors l₁ and l₂. The variables (A,B), (C, D) represent the signals from the two experiments, where the brackets indicate results from a single well.

I=I₀+ΔI±δi

R=R₀±δr

R₀=βI₀

A=l₁I B=l₂R

C=l₁R D=l₂I

Once those signals have been obtained, the ratio of the total signal can be found; in order to avoid confusing with the variable R, these fractions have been named f₁, f₂, f₃. Using Taylor expansion of the fractions and disregarding terms of second order or higher Õ, there are three expressions that allow the independent determination of the quantity of interest, ΔI/I₀.

$${f}_{1} = \frac{A}{B} = \frac{{l}_{1}}{{l}_{2} β} ( 1 \pm \frac{δ {i}^{A}}{{I}_{0}} \pm \frac{δ {r}^{B}}{β {I}_{0}} ) + \frac{{l}_{1}}{{l}_{2} β} \frac{Δ I}{{I}_{0}} + \tilde{O}$$

$${f}_{2} = \frac{C}{D} = \frac{{l}_{1} β}{{I}_{2}} ( 1 \pm \frac{δ {i}^{C}}{{I}_{0}} \pm \frac{δ {r}^{O}}{β {I}_{0}} ) + \frac{{l}_{1} β}{{I}_{2}} \frac{Δ I}{{I}_{0}} + \tilde{O}$$

$${f}_{3} = \frac{{f}_{1}}{{f}_{2}} = \frac{1}{{β}^{2}} ( 1 \pm \frac{δ {i}^{A}}{{I}_{0}} \pm \frac{δ {r}^{B}}{β {I}_{0}} \pm \frac{δ {i}^{C}}{{I}_{0}} \pm \frac{δ {r}^{D}}{β {I}_{0}} ) + \frac{2}{{β}^{2}} \frac{Δ I}{{I}_{0}} + \tilde{O}$$

$$\tilde{O} ≡ o ( \frac{δ {i}^{2}}{{i}_{0}^{2}} ) + o ( \frac{δ {r}^{2}}{{I}_{0}^{2}} ) + o ( \frac{δ i δ r}{{I}_{0}^{2}} ) + o ( \frac{δ i Δ I}{{I}_{0}^{2}} ) + o ( \frac{δ r Δ I}{{I}_{0}^{2}} ) + o ( \frac{Δ {I}^{2}}{{I}_{0}^{2}} )$$

When using several independent samples from multiple non-trisomy pregnancies, there are three variables to determine, namely l₁, l₂, β. Since there are three linearly independent equations, those three parameters can be determined. As a result, these parameters can be used in the analysis of the suspected trisomy samples.

$${f}_{1}^{′} = \frac{A}{B} = \frac{{l}_{1}}{{l}_{2} β} ( 1 \pm \frac{δ i}{{I}_{0}} \pm \frac{δ r}{β {I}_{0}} ) + \tilde{O} \to \frac{{l}_{1}}{{l}_{2} β}$$

$${f}_{2}^{′} = \frac{C}{D} = \frac{{l}_{1} β}{{l}_{2}} ( 1 \pm \frac{δ i}{{I}_{0}} \pm \frac{δ r}{β {I}_{0}} ) + \tilde{O} \to \frac{{l}_{1} β}{{l}_{2}}$$

$${f}_{3}^{′} = \frac{1}{{β}^{2}} ( 1 \pm \frac{δ {i}^{A}}{{I}_{0}} \pm \frac{δ {r}^{B}}{β {I}_{0}} \pm \frac{δ {i}^{C}}{{I}_{0}} \pm \frac{δ {r}^{D}}{β {I}_{0}} ) + \tilde{O} \to \frac{1}{{β}^{2}}$$

Three dyes per sample

An alternative idea to splitting the sample (as required for the dye-swap idea) is to include two reference probe sets and the probe set of interest.

Using a similar nomenclature to the dye swap equations, we have

I=I₀+ΔI±δi

R₁=R₁⁰±δr R₁⁰=β₁I⁰

R₂=R₂⁰±δr R₂⁰=β₂I⁰

The three signals from such an experiment are related to three labels with factors l₁, l₂, l₃.

A=l₁l

B=l₂R₁

C=l₃R₂

The three fractions are then

$${f}_{1} = \frac{A}{B} = \frac{{l}_{1}}{{l}_{2} {β}_{1}} ( 1 \pm \frac{δ {i}^{A}}{{I}_{0}} \pm \frac{δ {r}_{1}^{B}}{{β}_{1} {I}^{0}} ) + \frac{{l}_{1}}{{l}_{2} {β}_{1}} \frac{Δ I}{{I}^{0}}$$

$${f}_{2} = \frac{A}{C} = \frac{{l}_{1}}{{l}_{3} {β}_{2}} ( 1 \pm \frac{δ {i}^{A}}{{I}_{0}} \pm \frac{δ {r}_{2}^{C}}{{β}_{2} {I}^{0}} ) + \frac{{l}_{1}}{{l}_{3} {β}_{2}} \frac{Δ I}{{I}^{0}}$$

$${f}_{3} = \frac{B}{C} = \frac{{l}_{2} {β}_{1}}{{l}_{3} {β}_{2}} ( 1 \pm \frac{δ {r}_{1}^{B}}{{β}_{1} {I}^{0}} \pm \frac{δ {r}_{2}^{C}}{{β}_{2} {I}^{0}} )$$

where the second order Taylor expansion terms Õ have again been neglected.

From confirmed non-trisomy pregnancies, the limits for low noise are:

$${lim}_{δ \to 0} {f}_{1}^{′} = \frac{{l}_{1}}{{l}_{2} {β}_{1}}$$

$${lim}_{δ \to 0} {f}_{2}^{′} = \frac{{l}_{1}}{{l}_{3} {β}_{2}}$$

$${lim}_{δ \to 0} {f}_{3}^{′} = \frac{{l}_{2} {β}_{1}}{{l}_{3} {β}_{2}}$$

This means that there are three linearly independent equations and three parameters (or rather, combined parameters) l₁, l₂β₁, l₃β₂ that can be determined as a result. These are then useful for the data analysis in case of suspected trisomy samples. The additional advantage of this type of analysis using three different labels and two reference sets is that the noise of two similar sources can be quantified using fraction f₃ and its disomy cohort limit.

### Example 11

### Application to Single Cell Analysis

In order to predict signal intensities from 1 cell, such as for a PGD application with the method of the invention, we take into account existing data from model experiments. Here, 5 ng of input DNA gave a background subtracted average pixel intensity of 110 AFU when used with approximately 330,000 different labelled probe sequences (6 libraries) over an area of 7×10 ⁶ μm². This amount of DNA represents approximately 5 ng=5000 pg/6.6 pg/cell=758 cells. Under the assumption that the signal levels are to be maintained in the PGD application, certain predictions can be made.

| | Existing data | Extrapolation to PGD |
|---|---|---|
| BKGD sub signal intensity | 110 | 110 |
| Equivalent number of cells | 758 | 1 |
| Area of capture (um²) | 7000000 | 2308 |
| Labelled libraries | 6 | 1.5 |
| Labels per bait | 3 | 3 |

Using a with an average of 3 labels per bait, hybridising the genome from one cell to a circle of diameter 50 um (an area of 2308 um²) would yield average pixel intensities of 110 for disomy and 165 for trisomy without amplification.

It is likely that the average pixel intensity (or the capture area) could relatively easily be increased by increasing the labels/molecule (e.g. use of cy labels in addition to Kreatech labelling).

The following assumptions are made in this calculation;
No amplification is required
The amount of DNA in a cell is 6.6 pg
There is 100% yield from DNA purification and poly A tailing.
The yield from hybridisation of the baits and capture to dT is assumed to be the same as that Example 8.3

### Example 12

### Processing of Two Clinical Pregnancy Samples Using the Labelled Chromosome Baits and dT Slide Method

### Introduction

Clinical maternal plasma samples from a fetal disomy and a fetal trisomy pregnancy were received. A blinded experiment was carried out to identify the fetal chromosome 21 ploidy of the samples from circulating free DNA. DNA was extracted from 5 mls plasma and then amplified. An aliquot of each amplified sample was tagged with poly A, hybridised with differentially labelled chromosome 21 (test) and chromosome 18 (control) bait sets and captured on a dT slide via the poly A tag. The slide was then scanned on a microarray scanner and the relative fluorescent signals from the bait sets were used to infer the fetal chromosome 21 ploidy of the samples.

### Findings

The trisomy and disomy samples were correctly identified by comparing the ratio of the chromosome 21/chromosome 18 ratios of the two samples.

### Methods

DNA extraction and Amplification
Using the QIAamp Circulating Nucleic Acid Kit (Qiagen) DNA was extracted from 5 mls of the plasma samples, referred to below as sample 1 and sample 2, according to the manufacturer's instructions and eluted in 100 μl nuclease-free water
The samples were made up to 130 μl with Low TE and sheared on the Covaris AFA (Adaptive Focused Acoustics) Technology S2 ultrasonicator Machine with 6×1 min runs as recommended, followed by drying down on the Centrifugal concentrator SPD SpeediVac (ThermoSavant) for amplification
The samples were amplified using the GenomePlex kit (Sigma) according to the manufacturers instructions
   DNA tailing and Purification
80 ng (eight replicates of 10 ng) of both samples were phosphatase treated using antarctic phosphatase (NEB) in a 10 μl reaction volume, according to the manufacturer's instructions.
The samples were then tailed with terminal transferase (NEB) in a 20 μl reaction volume using a 1:5000 3′ end concentration:dATP. Four of the eight reactions per sample were stopped with EDTA after 10 mins. Reactions which were and were not stopped with EDTA are referred to as stopped and non-stopped samples respectively.
A-tailed samples were purified using 40 μl dT beads (Dynabeads Oligo (dT)25, Life Technologies) according to the manufacturers instructions and eluted in 6 μl water
0.5 μl was run on an R6K High Sensitivity RNA ScreenTape (Agilent)
The remainder was put into the bait hybridisation (see next step)

### DNA Bait Hybridisation and Capture to dT Slide

dT slide was generated as described previously
SureSelect XT custom baits (Agilent) were labelled using the Cy5 and Cy3 ULS labelling kit (Kreatech), and then hybridised to dT beads (Life Technologies) to remove sequences that would generate non-specific signal.
Tailed DNAs were mixed with 12 ng cy5 labelled chromosome 21, and 12 ng cy3 labelled chromosome 18 baits, 8 μl cot1DNA (1 μg/μl), 13 μl SureSelect hybridisation buffer (Agilent) to 24 μl. Labelled bait only control containing cot1 DNA was also set up.
Samples were heated to 95° C. for 5 mins and then cooled to 65° C.
1 μl Rnase Inhibitor (SureSelect kit) was added to each tube and the tubes incubated at 65° C. for 20 hours
Eight replicates of 3 μl aliquots were loaded at 65° C. into the 3 mm wells (CultureWells Grace Biolabs) on a dTslide (prewarmed to 40° C. on a hotblock) and covered with 6 μl mineral oil (Sigma. Molecular Biology grade)
The slide was incubated at 40° C. for two hours to allow capture of the tagged target molecules
The slide was submerged in wash 1(6×SSPE, 0.01% NLS) at 25° C. The sample and oil was flushed out of the wells by pipetting. The slide was washed for 10 mins in wash 1 at 25° C. followed by 5 mins in wash 2 (0.06×SSPE, 0.01% NLS) at 25° C.
The slide was scanned on an Agilent microarray scanner
The feature extraction was done using Genepix 6.0 software using a set of manually set-up features.
Data analysis was performed in excel (see next step)

### Data Analysis—Using the Ratio of Ratios to Determine the Trisomy Sample

The stopped and non-stopped tailing samples were analysed separately.
For each well, the ratio of the intensities of the red and green fluorescence values (chromosome 21/chromosome 18) was calculated. For each well, this quantity is referred to as the R-ratio.
In the case of a trisomy fetus, the R ratio will yield a greater value relative to a disomy fetus (the extent of which will be determined by the percentage fetal content in the maternal sample)
To determine if there is a difference in ploidy between samples 1 and 2, one R-ratio is divided by the other to generate a ratio of ratios. A deviation from 1 indicates a change in the ploidy of one of the samples and the magnitude of the value is dependant on the percentage foetal content

### Results and Discussion

### See FIG. 22 ScreenTape Analysis of Tailed Samples

The concentration of sample 1 after amplification and tailing was higher than that of sample 2.

The average length and molar concentrations of non-stopped samples is 1103nt (27.4 fmol/μl) and 1379nt (15.6 fmol/μl) for samples 1 and 2 respectively.

The average length and molar concentrations of the stopped samples is 347nt (23.5 fmol/μl) and 387nt (13.0 fmol/μl) for samples 1 and 2 respectively.

### Layout of the Slide (see FIG. 23)

The experiment includes eight replicates of each sample. Samples were arranged such that replicates were located at the edge as well as the middle of the slide.

Samples were prepared with both short (stopped) and long (non-stopped) poly A tails. A bait only negative control containing cot1 DNA was also included.

### Image of the Slide (Agilent Microarray Scanner—See FIG. 24)

Samples are arranged in columns of quadruplicates with two columns for each sample (8 wells in total)
sample1 not stopped and sample2 not stopped represent the samples with the standard full length tails
sample1 stopped and sample 2 stopped represent the samples with short poly A tails
Baits only contain only cot1 DNA with no tailed target
Features outlined with a red box were excluded from the analysis due to debris or other non-specific signal within the feature

### Mean of Median Raw Integrated Pixel Intensities—See FIG. 25

The plot shows the mean of median signal intensity for the cy5 labelled chromosome 21 and cy3 labelled chromosome 18 captured molecules, for each column of features across the slide; samples 1 and 2; poly A tailing not-stopped (ns) and stopped (s) and baits only.
The results indicate little difference in terms of signal intensity for the short and long poly A tails
A relatively high signal is observed for the negative control containing only the bait and the untailed cot1DNA

Calculation of the Ratio of R-Ratios (See FIG. 26)
Non-stopped and stopped samples were treated separately
The R ratio of sample 2 is divided by the R ratio of sample 1 for adjacent wells only
This ratio of R ratios>1 indicates sample 2 is trisomy
This ratio of R ratios<1 indicates sample 1 is trisomy
All samples indicate that sample 2 is a case trisomy 21
Samples with stopped tails(s) give more consistent ratios than non-stopped (ns). This observation could be explained by differences in the tail length of the samples. The ScreenTape analysis shows that the molar amount of purified stopped samples was less than that of non-stopped samples. Increased off-target hybridisation of baits resulting from the increased sample concentration and tail length of non-stopped samples could have depleted the bait to an extent that it is no longer in sufficient excess of the target.

### Cross-Referencing the Data Points (See FIG. 27)

The integrity of the data of the “stopped tailing” reactions was checked by cross referencing all of the sample 1 and sample 2 features. Ratio of ratios were calculated for all features (excluding 1 feature) in columns 3, 4, 8 and 9 of the slide (7×sample 1 wells and 8×sample 2 wells). The mean and standard deviation of the ratio of ratios are plotted.

In this plot trisomy/disomy is a representation of the mean of the sample 2/sample 1 ratios and disomy is the mean of sample 1/sample 1. The data strongly suggests that sample 2 is the trisomy sample. Calculation of the foetal content from the ratios suggest that the fetal content is approximately 24% of the total cell-free circulating DNA. As the raw data, with no background subtraction, was used in the analysis it is possible that this does not represent the true foetal content.

The data table shows the consistency of the technical replica, and cross-references samples 1/1, 1/2, and 2/2. The column headers in bold display the numbers that are the test/control ratios for each sample.

| | | sample 2 | | | | | | | | | | |
|---|---|---|---|---|---|---|---|---|---|---|---|---|
| | | 1.763 | 1.744 | 1.725 | 1.718 | 1.732 | 1.757 | 1.784 | 1.718 | mean | median | std |
| sample 1 | 1.531 | 1.15 | 1.14 | 1.13 | 1.12 | 1.13 | 1.15 | 1.17 | 1.12 | 1.14 | 1.14 | 0.02 |
| | 1.551 | 1.14 | 1.12 | 1.11 | 1.11 | 1.12 | 1.13 | 1.15 | 1.11 | 1.12 | 1.12 | 0.02 |
| | 1.609 | 1.10 | 1.08 | 1.07 | 1.07 | 1.08 | 1.09 | 1.11 | 1.07 | 1.08 | 1.08 | 0.01 |
| | 1.518 | 1.16 | 1.15 | 1.14 | 1.13 | 1.14 | 1.16 | 1.18 | 1.13 | 1.15 | 1.14 | 0.02 |
| | 1.565 | 1.13 | 1.11 | 1.10 | 1.10 | 1.11 | 1.12 | 1.14 | 1.10 | 1.11 | 1.11 | 0.02 |
| | 1.542 | 1.14 | 1.13 | 1.12 | 1.11 | 1.12 | 1.14 | 1.16 | 1.11 | 1.13 | 1.13 | 0.02 |
| | 1.587 | 1.11 | 1.10 | 1.09 | 1.08 | 1.09 | 1.11 | 1.12 | 1.08 | 1.10 | 1.09 | 0.01 |
| | 1.565 | 1.13 | 1.11 | 1.10 | 1.10 | 1.11 | 1.12 | 1.14 | 1.10 | 1.11 | 1.11 | 0.02 |
| | mean | 1.13 | 1.12 | 1.11 | 1.10 | 1.11 | 1.13 | 1.14 | 1.10 | 1.12 | | |
| | median | 1.13 | 1.12 | 1.11 | 1.10 | 1.11 | 1.13 | 1.14 | 1.10 | | 1.12 | |
| | std | 0.02 | 0.02 | 0.02 | 0.02 | 0.02 | 0.02 | 0.02 | 0.02 | | | 0.02 |
| | | sample 1 | | | | | | | | | | |
| | | 1.531 | 1.551 | 1.609 | 1.518 | 1.565 | 1.542 | 1.587 | 1.565 | mean | median | std |
| sample 1 | 1.531 | | 0.99 | 0.95 | 1.01 | 0.98 | 0.99 | 0.96 | 0.98 | 0.98 | 0.98 | 0.02 |
| | 1.551 | 1.01 | | 0.96 | 1.02 | 0.99 | 1.01 | 0.98 | 0.99 | 0.99 | 0.99 | 0.02 |
| | 1.609 | 1.05 | 1.04 | | 1.05 | 1.03 | 1.04 | 1.01 | 1.03 | 1.04 | 1.04 | 0.02 |
| | 1.518 | 0.99 | 0.98 | 0.94 | | 0.97 | 0.98 | 0.96 | 0.97 | 0.97 | 0.97 | 0.02 |
| | 1.565 | 1.02 | 1.01 | 0.97 | 1.03 | | 1.02 | 0.99 | | 1.01 | 1.01 | 0.02 |
| | 1.542 | 1.01 | 0.99 | 0.96 | 1.02 | 0.98 | | 0.97 | 0.98 | 0.99 | 0.98 | 0.02 |
| | 1.587 | 1.04 | 1.02 | 0.99 | 1.05 | 1.01 | 1.03 | | 1.01 | 1.02 | 1.02 | 0.02 |
| | 1.565 | 1.02 | 1.01 | 0.97 | 1.03 | 1.00 | 1.02 | 0.99 | | 1.01 | 1.01 | 0.02 |
| | mean | 1.02 | 1.01 | 0.96 | 1.03 | 1.00 | 1.01 | 0.98 | 0.99 | 1.00 | | |
| | median | 1.02 | 1.01 | 0.96 | 1.03 | 0.99 | 1.02 | 0.98 | 0.99 | | 1.00 | |
| | std | 0.02 | 0.02 | 0.01 | 0.02 | 0.02 | 0.02 | 0.02 | 0.02 | | | 0.03 |
| | | sample 2 | | | | | | | | | | |
| | | 1.763 | 1.744 | 1.725 | 1.718 | 1.732 | 1.757 | 1.784 | 1.718 | mean | median | std |
| sample 2 | 1.763 | | 1.01 | 1.02 | 1.03 | 1.02 | 1.00 | 0.99 | 1.03 | 1.01 | 1.02 | 0.01 |
| | 1.744 | 0.99 | | 1.01 | 1.01 | 1.01 | 0.99 | 0.98 | 1.01 | 1.00 | 1.01 | 0.01 |
| | 1.725 | 0.98 | 0.99 | | 1.00 | 1.00 | 0.98 | 0.97 | 1.00 | 0.99 | 0.99 | 0.01 |
| | 1.718 | 0.97 | 0.99 | 1.00 | | 0.99 | 0.98 | 0.96 | | 0.98 | 0.98 | 0.01 |
| | 1.732 | 0.98 | 0.99 | 1.00 | 1.01 | | 0.99 | 0.97 | 1.01 | 0.99 | 0.99 | 0.01 |
| | 1.757 | 1.00 | 1.01 | 1.02 | 1.02 | 1.01 | | 0.98 | 1.02 | 1.01 | 1.01 | 0.01 |
| | 1.784 | 1.01 | 1.02 | 1.03 | 1.04 | 1.03 | 1.02 | | 1.04 | 1.03 | 1.03 | 0.01 |
| | 1.718 | 0.97 | 0.99 | 1.00 | 1.00 | 0.99 | 0.98 | 0.96 | | 0.98 | 0.99 | 0.01 |
| | mean | 0.99 | 1.00 | 1.01 | 1.02 | 1.01 | 0.99 | 0.97 | 1.02 | 1.00 | | |
| | median | 0.98 | 0.99 | 1.01 | 1.01 | 1.01 | 0.99 | 0.97 | 1.02 | | 1.00 | |
| | std | 0.01 | 0.01 | 0.01 | 0.01 | 0.01 | 0.01 | 0.01 | 0.01 | | | 0.02 |

### Probability Density Function (FIG. 28)

The separation between the two samples, taken from the 15 data points used above is illustrated in the probability density.

### Conclusion

In a blinded experiment of two pregnancy plasma samples containing cell-free DNA, the correct sample was identified as fetal trisomy. The result was confirmed by cross-referencing the datapoints.

1. A method for the measurement of the amount or difference in the amounts of 2 or more nucleic acid targets in a sample, the method comprising the steps of attaching to nucleic acids present in the sample:
a tag which allows the nucleic acids to be captured to a solid support; and
a labelled probe for a first nucleic acid target present in the sample and a labelled probe for a second nucleic acid target present in the sample, and then measuring the amount of each labelled probe or difference in the amount of labelled probes;
wherein the probe is not a single labelled nucleotide.

2. A method according to claim 1, wherein the sample comprises nucleic acid derived from the blood or urine of a pregnant female, or from an individual being assessed for cancer, or from a cell of a blastocyst, or from an individual who has received an organ transplant, or from an individual who has, or is being assessed for the presence of, a disorder or disease associated with a change, such as a duplication or deletion, in the amount of a first nucleic acid target in a genome compared with a normal individual.

3. A method according to claim 1, wherein the first and second nucleic acid targets are located on different chromosomes.

4. A method according to claim 3, wherein the first probe is for a target nucleic acid sequence associated with aneuploidy and the second probe is for target nucleic acid not associated with aneuploidy.

5. A method according to claim 4, wherein the first probe is for human chromosome 21, 13, or 18.

6. A method according to claim 1, wherein the first probe is for a target nucleic acid sequence associated with a cancer and the second probe is for target nucleic acid not associated with cancer.

7. A method according to claim 1, wherein the first probe is for a target nucleic acid sequence associated with a specific mRNA and the second probe is for target nucleic acid not associated with that mRNA.

8. A method according to claim 1, wherein the first and second probes comprise fluorescent labels with distinguishable emission spectra and the amount of a probe, or the difference between the probes, is measured by fluorescence.

9. A method according to claim 1, wherein the measurement occurs at the level of individual probes.

10. A method according to claim 1, wherein the measurement occurs across the population of labelled probes for the first target and/or across the population of probes of the second target.

11. A method according to claim 1, wherein the probes attached to their target nucleic acid are captured on a solid support before measuring the amount of labelled probe or difference in amount of labelled probe.

12. A method according to claim 1, wherein the first and second probes each comprise a first section of sequence complementary to a target nucleic acid, and each further comprise a second section that can be used to differentiate the first and second probes.

13. A method according to claim 12, wherein the second sections of the first and second probes are different from one another and are captured by capture agents bound to a solid support.

14. A method according to claim 12, wherein the second sections of the first and second probes are different from one another and the second section acts as a modulator of mobility during electrophoresis such that the first and second probes may be discriminated by differential mobility during electrophoresis.

15. A method according to claim 12, wherein the second section of the first and second probes are complementary in sequence, such that they can hybridise with one another, and wherein the first and second probe are labelled with a fluorophore and with a quenching agent, respectively, such that hybridisation of the complementary sequences of the first and second probes brings the quencher and fluorophore into juxtaposition such that quenching of the fluorophore can take place on juxtaposed probes.

16. A method according to claim 1, wherein nucleic acids in the sample are tagged to oligonucleotides which permit amplification (such as by the polymerase chain reaction) and which further permit attachment to a solid support derivatised with oligonucleotides of complementary sequence.

17. A method according to claim 16, wherein nucleic acids in the sample are amplified by the polymerase chain reaction after which the amplification products are denatured and hybridised with libraries of labelled target specific probes.

18. A method according to claim 1, comprising a first set of labelled probes for a first nucleic acid target and a second set of labelled probes for a second nucleic acid target, wherein the first and second sets contain multiple different probes for the first and second nucleic acid targets respectively.

19. A method according to claim 18, wherein each probe within a probe set comprises a first section of sequence complementary to a target nucleic acid, and a second section of sequence that is the same within all members of the set that can be used to differentiate the first and second probe sets.

20. A method according to claim 1, comprising a probe or probe set for an additional target or targets.

21. A method according to claim 20, wherein the additional target is a second control and comparison between two controls can provide an internal measurement on the degree of error.

22. A method according to claim 1, for identification of a woman carrying a fetus with aneuploidy.

23. A method according to claim 1, wherein the amount of label is determined by pixel intensity and/or pixel number and the method comprises a step of identification and exclusion of the pixel outliers from the data analysis.

24. A method according to claim 1, wherein the sample is divided into a first and second aliquot, wherein the first aliquot is probed with a first probe labelled with first label for a first nucleic acid target present in the sample and with a second probe labelled with a second (different) label for a second nucleic acid target present in the sample;
wherein the second aliquot is probed with the first probe labelled with the second label for a first nucleic acid target present in the sample and a second probe labelled with the first label for a second nucleic acid target present in the sample.

25. A method according to claim 1, wherein a first probe set comprises probes specific to sequences on two or more chromosomes and a second probe set comprises probes specific to sequences on two or more chromosomes which are different from the chromosomes to which the first probes are specific, optionally wherein the set of probes is designed to cover the whole genome, excluding X and Y chromosomes, suitably where each chromosome is represented by the same number of probes.

26. A kit comprising: a probe or probe set for a first nucleic acid and probe or probe set for a second nucleic acid, wherein the first probe or probe set is for a nucleic acid target associated with a disorder and a second probe or probe set is for a nucleic acid target not associated with the disorder, wherein the disorder is associated with a change in the amount of the first nucleic acid target in a genome.

27. A kit comprising: a tag that may be attached to a nucleic acid to allow that nucleic acids to be captured to a solid support and a probe or probe set for a nucleic acid target associated with a disorder, the disorder being associated with a change in the amount of nucleic acid target in a genome, such as aneuploidy.

28. A kit according to claim 26, comprising an additional probe or probe set against an additional target or targets.

29. A method for the measurement of the differences in the amounts of 2 or more nucleic acid targets in a sample, the method comprising the steps of attaching to nucleic acids present in the sample:
a tag which allows the nucleic acids to be captured to a solid support; and
a probe for a first nucleic acid target present in the sample and a probe for a second nucleic acid target present in the sample,
wherein each probe comprises 2 primer portions, the primer portions differing between the 2 probes, and wherein the probe primers portions serve as targets for amplification primers to amplify the first and second probes, wherein the amplification reaction for the first and second probe uses a labelled amplification primer, and wherein the label for amplification of the first and second probe is different such that the product of the amplification of the first and second probe is a differently labelled amplification product.

30. A method or kit according to claim 1, wherein the tag is of a defined length or is within a defined range of fragment lengths.

31. A method according to claim 1, wherein the tag is a homopolymer tail added to nucleic acid in the sample, and the tailing reaction is terminated before the tail reaches the maximum tail length.

32. A method or kit according to claim 30, wherein the tag is a polyA tail of less than 1000 nucleotides in length.
